(19) United States
(12) Patent Application Publication
(10) Pub. No.: US 2022/0304606 A1
(43) Pub. Date: Sep. 29, 2022

(54) MAGNETIC MEASUREMENT APPARATUS AND MAGNETIC MEASUREMENT SYSTEM

(71) Applicants: Toshihiro ISHII, Miyagi (JP); Hirofumi MORISE, Kanagawa (JP)

(72) Inventors: Toshihiro ISHII, Miyagi (JP); Hirofumi MORISE, Kanagawa (JP)

(21) Appl. No.: 17/692,484

(22) Filed: Mar. 11, 2022

(30) Foreign Application Priority Data
Mar. 23, 2021 (JP) 2021-049287

(51) Int. Cl. A61B 5/245; G01R 33/09; A61B 5/00

(52) U.S. Cl. CPC A61B 5/245; G01R 33/09; A61B 5/6822; A61B 2562/0223

(57) ABSTRACT

A magnetic measurement apparatus includes a concentrating structure and a magnetic sensor. The concentrating structure includes a band portion and a plurality of protruding portions. The band portion is configured to concentrate a magnetic flux from a subject. The plurality of protruding portions are configured to transmit the concentrated magnetic flux to a magnetic sensor. The magnetic sensor is magnetically connected between two opposing protruding portions.

### CROSS-REFERENCE TO RELATED APPLICATIONS

The present application claims priority under 35 U.S.C. § 119 to Japanese Patent Application No. 2021-049287, filed on Mar. 23, 2021. The contents of which are incorporated herein by reference in their entirety.

### BACKGROUND OF THE INVENTION

### 1. Field of the Invention

The present invention relates to a magnetic measurement apparatus and a magnetic measurement system.

### 2. Description of the Related Art

An electroencephalography (EEG) as an apparatus that detects biological neural activities and a method of electrically measuring neurotransmission disorders in a spine, a carpal tunnel, a cubital region, and the like are known. For example, as a method called inching, a method of arranging a plurality of electrodes and calculating a position at which nerve block occurs is known. However, in the electrical measurement, it is difficult to calculate an accurate position of the nerve block. Further, in a conventional EEG apparatus, it is necessary to apply cream to a head portion to reduce interface resistance between the electrodes of the EEG apparatus and a scalp. Application of the cream causes a feeling of discomfort and it is necessary to wash the hair after the measurement is finished.

In contrast, in magnetic field measurement, there is no large impedance at an interface between a living body and a sensor, so that it is possible to improve measurement accuracy and accurately calculate a position at which nerve block occurs. Further, it is possible to reduce a problem with misalignment due to mounting on the head portion and long-time measurement. However, in the magnetic field measurement, it is necessary to cool a superconducting quantum interference device (SQUID) with liquid helium (He), and it is necessary to fix a large dewar at a spinal portion or a head portion. Furthermore, in an ordinary temperature magnetic sensor, such as an optically pumped atomic magnetometer (OPM), it is necessary to perform measurement in a zero magnetic field, so that it is inevitable to perform measurement in a magnetic shielding room with high magnetic shielding performance. Moreover, it takes one million yen for a single OPM sensor, which is expensive.

A magneto resistive (MR) sensor is able to perform measurement under geomagnetism, but a noise floor of a current MR sensor is about 3 pT√Hz (1 Hz) or 400 fT√Hz (100 Hz). Intensity of a cervical spinal signal is several tens fT and intensity of a signal due to brain neural activities is several hundred fT. At the sensitivity as described above, it is difficult to detect a signal of a cervical spine or a head portion that is weaker than noise in the noise floor. In other words, the sensitivity of the MR sensor is inadequate.

To improve the sensitivity of the MR sensor, conventionally, a concentrating structure has been studied.

For example, Japanese Unexamined Patent Application Publication No. 2019-174140 discloses a structure in which two magnetic bodies are provided and a magnetic sensor element is arranged between the two magnetic bodies through which magnetic flux passes. In the concentrating structure, a pair of magnetic bodies is provided for a single sensor. Further, for example, U.S. Unexamined Patent Application Publication No. 2020/0057115 discloses a structure in which a sensor is arranged at an end portion of a magnetic body having a pyramid structure.

Furthermore, even if internal noise of the MR sensor as described above is reduced, there may be an influence of external noise, and a signal to be measured may be lost in the noise. Therefore, a method of implementing a gradiometer for removing noise by arranging a reference sensor and deducing external noise detected by the reference sensor from a signal of a detection sensor is adopted.

A general gradiometer adopts a method of arranging the reference sensor at a position separated by 30 millimeters (mm) from a subject relative to the detection sensor so as to distinguish between external noise and a biological signal. With the separation by 30 mm, for example, the reference sensor is separated by 50 mm from a sulcus (based on the assumption that the scalp—the sulcus is 20 mm), and assuming that a signal intensity ratio with respect to the detection sensor (arranged on the scalp) is proportional to 1/r\hat{ }2 based on the Lambert's law, the intensity ratio of about 0.4 is obtained. Assuming that the external noise is input at approximately the same intensity, an intensity ratio between a biological signal and the external noise is 0.4. However, with this level of the intensity ratio, it is difficult to fully achieve the effect of the gradiometer and this may cause an error. Further, in the gradiometer in which the distance of 30 mm is ensured, it is likely that equivalence of external noise is not retained. If external noise uniformity of even several % is observed, it is difficult to fully eliminate the noise.

For example, as indicated by Japanese Patent No. 5224486 or the like, a method of eliminating a second-order term of a magnetic field strength distribution is needed. A secondary component of the magnetic field strength distribution is not a linear and homogenous inclined magnetic field, but a non-linear and curved magnetic field strength distribution is obtained. Therefore, in the gradiometer in which the distance of 30 mm is ensured and in which a simple difference over the distance of 30 mm is obtained, an error remains. It is necessary to reduce the distance between the reference sensor and the detection sensor as much as possible, and at the same time, it is necessary to contrive ways to ensure the intensity ratio between the signal and the external noise (about 0.4 in the conventional example). It is necessary to reduce noise and improve sensitivity.

In the conventional technology, it is difficult to improve inadequate sensitivity of a magnetic sensor that is available under geomagnetism and at ordinary temperature, and it is difficult to capture a magnetic signal with high accuracy.

The present invention has been conceived in view of the foregoing situation, and an object of the present invention is to provide a magnetic measurement apparatus and a magnetic measurement system capable of improving inadequate sensitivity of a magnetic sensor that is available under geomagnetism and at ordinary temperature, and capturing a magnetic signal with high accuracy.

### SUMMARY OF THE INVENTION

According to an aspect of the present invention, a magnetic measurement apparatus includes a concentrating structure and a magnetic sensor. The concentrating structure includes a band portion and a plurality of protruding portions. The band portion is configured to concentrate a magnetic flux from a subject. The plurality of protruding portions are configured to transmit the concentrated magnetic flux to a magnetic sensor. The magnetic sensor is magnetically connected between two opposing protruding portions.

### BRIEF DESCRIPTION OF THE DRAWINGS

FIG. 1 is a diagram illustrating an example of the way to attach a magnetic measurement apparatus of a first embodiment;

FIG. 2 is a diagram illustrating an example of a fixing device of the magnetic measurement apparatus of the first embodiment;

FIG. 3 is a diagram illustrating positions of cervical vertebrae;

FIG. 4 is a diagram illustrating an example of the magnetic measurement apparatus of the first embodiment;

FIG. 5 is a diagram illustrating an example of an internal structure of the magnetic measurement apparatus of the first embodiment;

FIG. 6 is a developed view of a concentrating structure of the first embodiment;

FIG. 7 is a diagram illustrating an example of the concentrating structure in which a band portion is removed according to the first embodiment;

FIG. 8 is an enlarged view of two opposing protruding portions of the first embodiment;

FIG. 9 is a diagram illustrating an example of a magnetic simulation on a concave surface of the first embodiment;

FIG. 10 is a diagram illustrating an example of a connection portion between the protruding portions and the band portion of the first embodiment;

FIG. 11 is a diagram illustrating an example of a space and a protrusion length of the first embodiment;

FIG. 12 is a diagram illustrating an example of the opposing protruding portions and an interval (gap) between the protruding portions of the first embodiment;

FIG. 13 is a diagram illustrating an example of unevenness on the protruding portions of the first embodiment;

FIG. 14 is a diagram illustrating an example of roughness of a protrusion end and surface roughness of the first embodiment;

FIG. 15 is a diagram illustrating an example of a sensor configuration of a single unit of the first embodiment;

FIG. 16 is a diagram illustrating, by image, functions of the concentrating structure of the first embodiment;

FIG. 17 is a diagram for explaining an example of a simulation (example of a calculation model) of the concentrating structure of the first embodiment using the finite element method;

FIG. 18 is a diagram for explaining the example of the simulation (example of the calculation model) of the concentrating structure of the first embodiment using the finite element method;

FIG. 19A is a diagram illustrating a magnetic flux density when lengths (X direction) are changed;

FIG. 19B is a diagram illustrating the magnetic flux density when widths (Y direction) are changed;

FIG. 19C is a diagram illustrating the magnetic flux density when thicknesses (Z direction) are changed;

FIG. 20 is a diagram illustrating an example of a configuration of a magnetic measurement system of the first embodiment;

FIG. 21 is a flowchart illustrating an example of a measurement method of the first embodiment;

FIG. 22A is a diagram illustrating a mounting example of markers of the first embodiment;

FIG. 22B is a diagram illustrating the mounting example of the markers of the first embodiment;

FIG. 23 is a diagram for explaining brain functions and areas;

FIG. 24 is a diagram for explaining a direction in which a dipole occurs;

FIG. 25 is a diagram illustrating an example of arrangement of a concentrating structure of a second embodiment;

FIG. 26 is a diagram illustrating an example of arrangement of the concentrating structure of the second embodiment;

FIG. 27 is a diagram illustrating an example of arrangement of the concentrating structure of the second embodiment;

FIG. 28 is a diagram illustrating an angle formed by two protruding portions of the second embodiment;

FIG. 29 is a diagram illustrating a relationship between the angle formed by the two protruding portions of the second embodiment and magnetic flux intensity;

FIG. 30 is a flowchart illustrating an example of a measurement method of the second embodiment;

FIG. 31 is a diagram illustrating a position of a carpal tunnel;

FIG. 32 is a schematic diagram illustrating a first example of a magnetic measurement apparatus of a third embodiment;

FIG. 33 is a schematic diagram illustrating a second example of the magnetic measurement apparatus of the third embodiment;

FIG. 34 is a diagram illustrating an example of a magnetic shielding box (MSB) for a brachium of the third embodiment;

FIG. 35 is an image illustrating an example of a measurement result of the third embodiment;

FIG. 36 is an image of an example of a measurement result (carpal tunnel syndrome) of the third embodiment;

FIG. 37 is a diagram illustrating an example of measurement for cubital tunnel syndrome according to a fourth embodiment;

FIG. 38 is an image illustrating an example of a measurement result of the fourth embodiment;

FIG. 39 is a diagram illustrating an example of measurement in a nerve conduction study (diabetes) according to a fifth embodiment;

FIG. 40 is an image illustrating an example of a measurement result of the fifth embodiment;

FIG. 41 is a diagram illustrating a magnetic flux distribution in a case where the concentrating structures of the first and the second embodiments are adopted;

FIG. 42 is a diagram illustrating a magnetic flux distribution in a case where the concentrating structures of the first and the second embodiments are not adopted; and

FIG. 43 is a diagram illustrating an example of a hardware configuration of an information processing apparatus of the first and the second embodiments.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to provide a magnetic measurement apparatus and a magnetic measurement system capable of improving inadequate sensitivity of a magnetic sensor that is available under geomagnetism and at ordinary temperature, and capturing a magnetic signal with high accuracy.

A magnetic measurement apparatus according to the present application includes a concentrating structure that includes a band portion for concentrating magnetic flux from a subject and a plurality of protruding portions for transmitting the concentrated magnetic flux to a magnetic sensor, and includes a detection sensor that is magnetically connected between two opposing ones of the protruding portions. With this configuration, it is possible to concentrate the magnetic flux generated from a measurement portion of a subject, and transmit the concentrated magnetic flux to the detection sensor through the protruding portions. In this case, by arranging the detection sensor between the two opposing protruding portions, the magnetic flux that is preferentially transmitted to the opposing protruding portions passes through the detection sensor, so that it is possible to detect a magnetic signal in the measurement portion with high accuracy.

Embodiments of a magnetic measurement apparatus and a magnetic measurement system according to the present invention will be described in detail below with reference to the drawings. The present invention is not limited by the embodiments below. Structural elements in the embodiments described blow include one that can easily be thought of by a person skilled in the art, one that is practically identical, and one that is within an equivalent range. Further, within the scope not departing from the gist of the following embodiments, various omission, replacement, modifications, and combinations may be made.

### First Embodiment

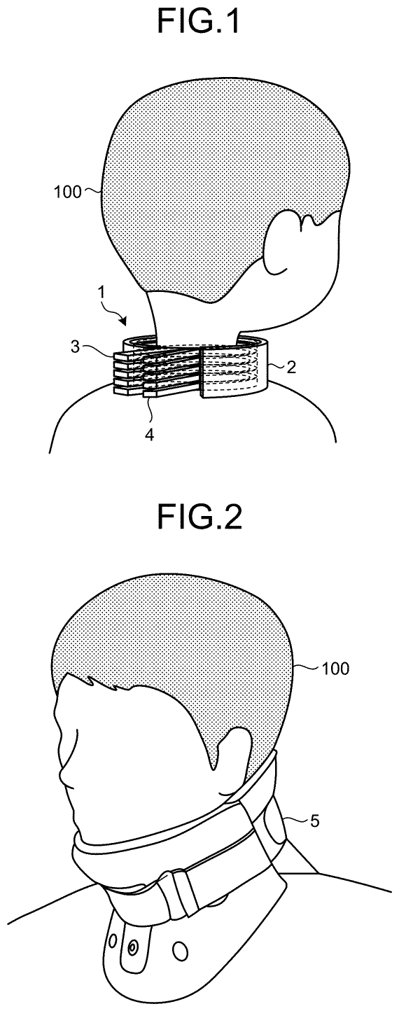

FIG. 1 is a diagram illustrating an example of the way to attach a magnetic measurement apparatus 1 of a first embodiment. The example in FIG. 1 illustrates the way to attach the magnetic measurement apparatus 1 for a cervical spine (a magnetospinography in the first embodiment) of the present embodiment to a subject 100 (one example of a subject). FIG. 2 is a diagram illustrating an example of a fixing device 5 of the magnetic measurement apparatus 1 of the first embodiment. A fixing jig 2 of the first embodiment is in the form of a band like a corset, and is wound around and fixed to a neck of the subject 100 with the aid of the fixing device 5 as illustrated in FIG. 2, for example.

Detection sensors 3 and reference sensors 4 are mounted in a manner of being inserted in the fixing jig 2. The detection sensors 3 are magnetic sensors that are arranged on a back surface of the neck of the subject 100. In the first embodiment, magneto resistive (MR) sensors are adopted as the detection sensors 3. Response speed of the MR sensors is 1 kHz that corresponds to response of the cervical spine at ordinary temperature and under geomagnetism, so that it is possible to provide the high-accuracy magnetic measurement apparatus 1.

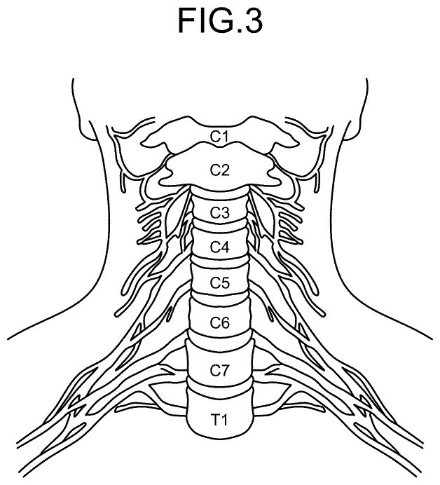

FIG. 3 is a diagram illustrating positions of cervical vertebrae C1 to C7. The detection sensors 3 are arranged so as to cover all of the positions of the cervical vertebrae C7 to C2. In the present embodiment, as illustrated in FIG. 1, the five detection sensors 3 are arranged at a pitch of about 30 mm. The number of the detection sensors 3 and the pitch may be appropriately changed by preparing the plurality of fixing jigs 2 and switching between the fixing jigs 2.

An outer layer of the fixing device 5 is made of resin and formed as a band to allow fixation. The fixing jig 2 (housing) that has a cylindrical shape and that has a magnetic shielding function is arranged inside the fixing device 5, and includes a mechanism that allows the detection sensors 3 to be attached and detached. A flexible cylindrical plate object with a thickness of about 4 mm is mounted, as a concentrating structure, in an inner layer so as to lightly press the neck of the subject 100. Sizes (a diameter and a length) of the cylindrical object of the concentrating structure are selected in accordance with a body shape of the subject 100.

In the first embodiment, a plurality of fixing jigs with different sizes are prepared in accordance with physical sizes of the subjects 100. For example, as for a length direction of the fixing jig 2, by preparing a plurality of kinds, such as five different lengths from about 5 centimeters (cm) to 15 cm with 2 cm increments, it is possible to cope with various persons from women with small physical sizes to men with large physical sizes. Further, as for a thickness direction corresponding to a dimeter of a neck, by preparing five different thicknesses for example, it is possible to reduce distances to cervical vertebrae in a cervical spine, so that it is possible to obtain an intensive signal due to a relationship between the distance and the signal intensity that is determined by the Biot-Savart's law.

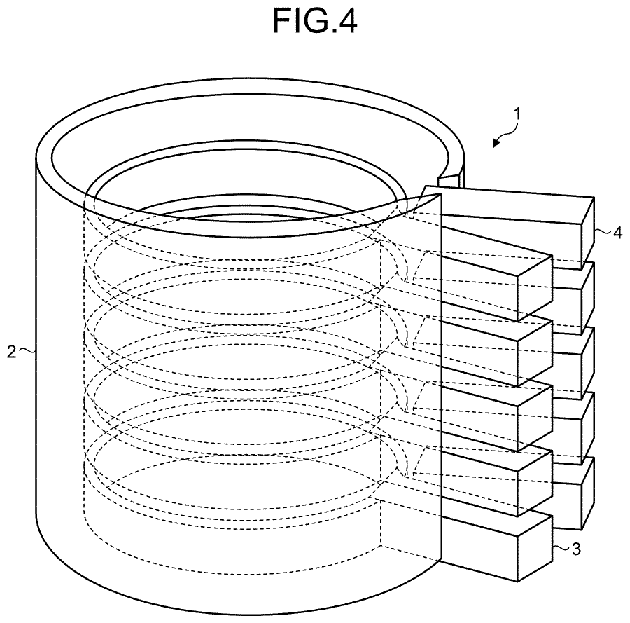

FIG. 4 is a diagram illustrating an example of the magnetic measurement apparatus 1 of the first embodiment. The magnetic measurement apparatus 1 of the first embodiment includes the fixing jig 2, the detection sensors 3, and the reference sensors 4. As illustrated in FIG. 4, the fixing jig 2 (housing) is made of a material (magnetic shielding member) that has a magnetic shielding function, and has a mechanism that allows the detection sensors 3 and the reference sensors 4 to be attached and detached.

The magnetic shielding member may be made of a metal material, such as general permalloy, but it is necessary to reduce a weight of the magnetic shielding member for mounting on the cervical spine or the like. A “ferrite particle” with high magnetic absorption, the permalloy, or the like is heavy in weight, and can hardly be held by a subject. A light weight and high magnetic shielding performance are needed. In general, a shielding material, such as FINEMET (trademark), made of an amorphous foil is distributed. The amorphous foil can be rapidly cooled from a molten state because it is thinned, and is cured in an amorphous state before metallic crystallization. A thickness of the amorphous foil is several tens microns, and both sides thereof are held by films. The amorphous film has high magnetic permeability exceeding 5000 based on conversion into a single thickness. Further, the amorphous film is extremely thin, and therefore is extremely light in weight as compared to permalloy that is a general shielding member, and can implement the equivalent shielding performance. Furthermore, the amorphous film is in the form of a film and therefore can easily be processed.

A material obtained by mixing ferrite particles (about 2 micrometers (um)) with resin is used as the magnetic shielding member of the present embodiment. General resin, such as polyethylene terephthalate (PET) or rubber, may be used. In the present embodiment, silicone resin that can easily contain particles and that has elasticity is used.

The silicone resin is formed into a film shape with a thickness of about 1 mm, and subsequently, a film of the amorphous metallic foil as described above is laminated. Four layers are laminated and a magnetic shield with a thickness of about 4.5 mm is formed. The fixing jig 2 (housing) that is made of the magnetic shielding member as described above has an annular shape as illustrated in FIG. 4, and the magnetic shielding member is intended to prevent an external noise magnetic field from entering the detection sensors 3.

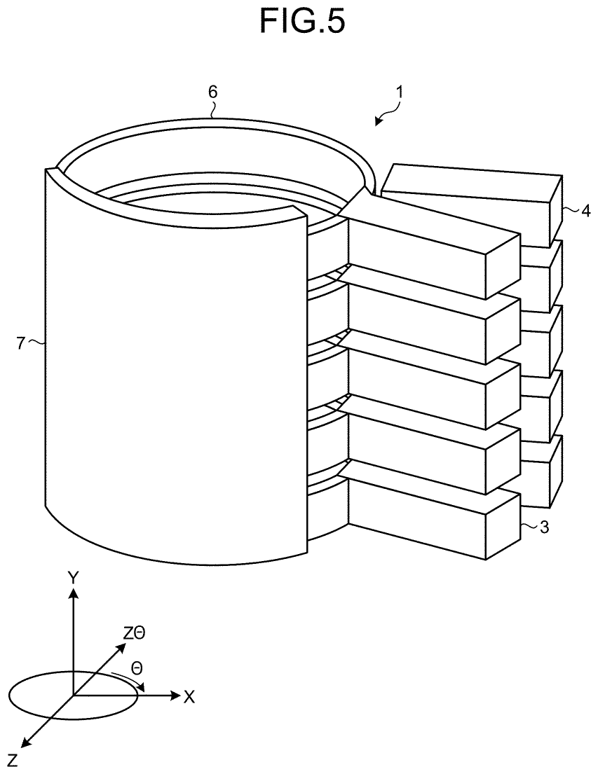

FIG. 5 is a diagram illustrating an example of an internal structure of the magnetic measurement apparatus 1 of the first embodiment. FIG. 5 illustrates an example of the internal structure in the case where the magnetic shielding member (the fixing jig 2) is detached. The concentrating structure includes protruding portions 6 and a band portion 7.

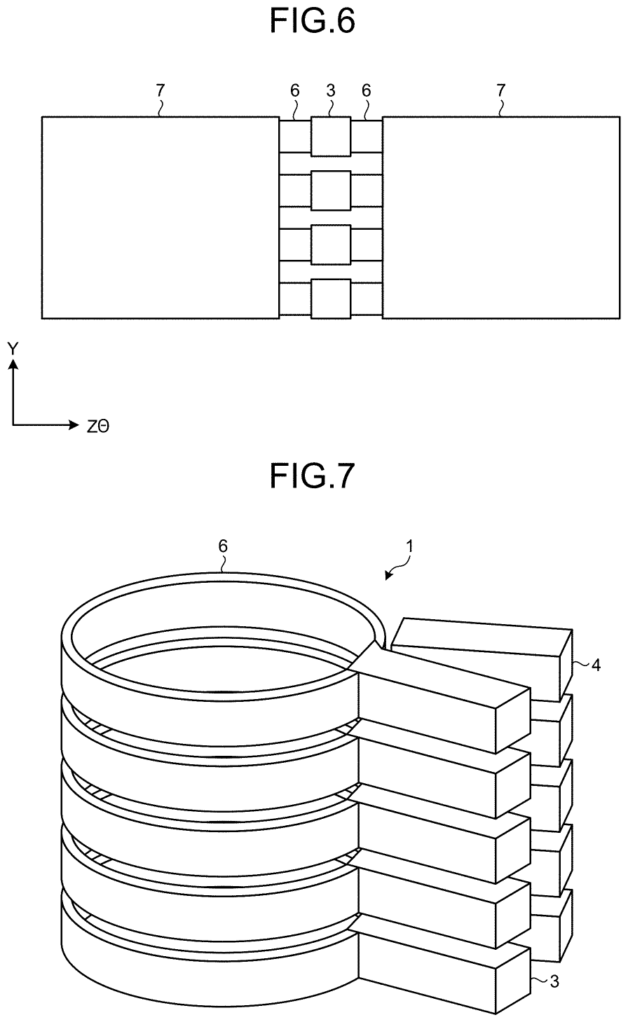

FIG. 6 is a developed view of the concentrating structure of the first embodiment. FIG. 6 illustrates a developed view of the concentrating structure that is wound around the cervical spine of the subject 100. As illustrated in FIG. 5 and FIG. 6, the protruding portions 6 are arranged in line in the Y direction, and the detection sensors 3 corresponding to the protruding portions 6 are arranged in the Y direction. The Y direction (first direction) corresponds to a neurotransmission direction in the cervical spine (spine) of the subject 100 in FIG. 1.

An electrical current flows (an electric current dipole is transmitted) through the spine, such as the cervical spine, for neurotransmission. When there is a defect in transmission of the electric current due to hernia or the like, it is possible to appropriately perform an operation on a portion if the position of the portion can be accurately determined in a non-invasive way. As illustrated in FIG. 6, by arranging the detection sensors 3 in the direction (Y direction) in which the electrical current is transmitted, it is possible to improve spatial resolution in this direction. Further, a magnetic field is generated in a direction (Zθ direction) perpendicular to the Y direction, and therefore, by arranging the protruding portions 6 in the perpendicular direction, it is possible to detect magnetic flux generated in this direction with high sensitivity.

FIG. 7 is a diagram illustrating an example of the concentrating structure in which the band portion 7 is removed according to the first embodiment. In the present embodiment, the protruding portions 6 and the band portion 7 are detachably attached, but the protruding portions 6 and the band portion 7 may be configured in an integrated manner.

Both of the band portion 7 and the protruding portions 6 are made of ferromagnetic bodies. In the present embodiment, for both of the band portion 7 and the protruding portions 6, a magnetic material in which an amorphous metallic foil is supported by a film is adopted as the ferromagnetic bodies. The amorphous foil is realized by a manufacturing method of rapidly cooling a molten metal, which is extended to have a thickness of several tens microns, with the aid of this thinness, and curing the metal before crystallization, and has magnetic permeability of more than 5000 by conversion. In the present embodiment, by laminating the amorphous foils, the magnetic shielding performance is improved. By ensuring a certain space, a design in which a skin effect is fully achieved is realized. Further, as will be described later, the amorphous foil is flexible and deformable because it has a film shape, and therefore can conform to a curved surface of the cervical spine or the like. In this example, FINEMET (registered trademark) is adopted. As for the number of laminated layers, even about 30 laminated layers have a thickness of about 3 mm, which is thin, and can realize the shielding performance as good as the permalloy with the thickness of 3 mm.

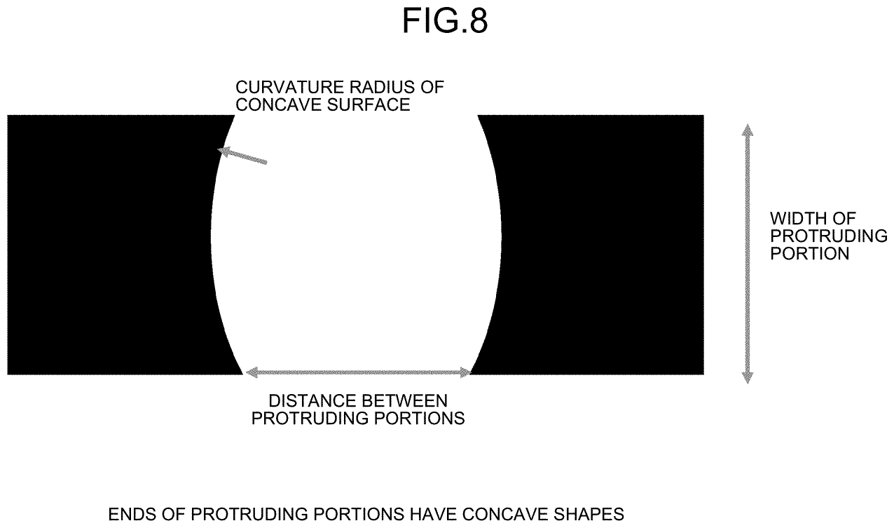

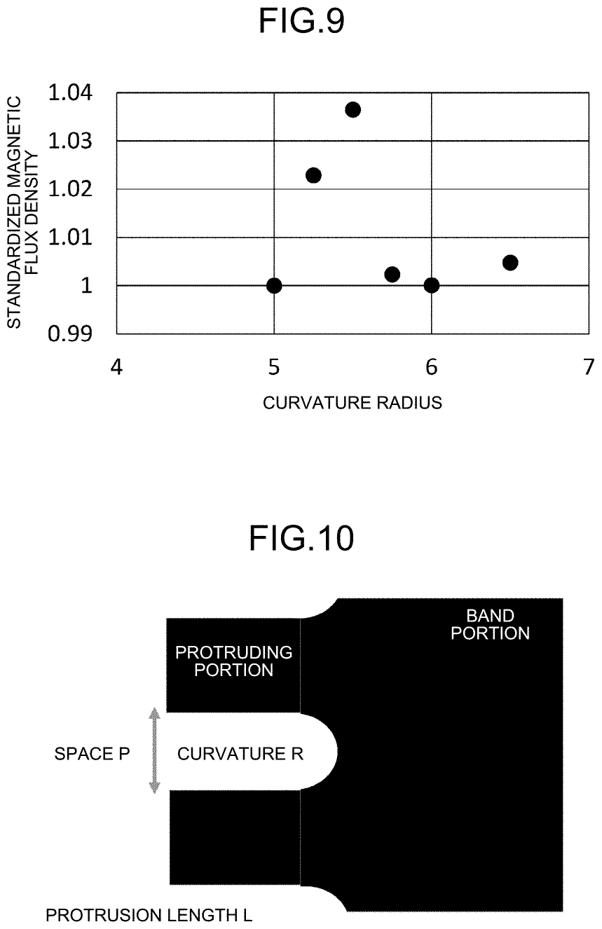

FIG. 8 is an enlarged view of the two opposing protruding portions. As illustrated in the figure, it is desirable that portions of the protruding portions facing the magnetic sensor have concave surfaces. This can be verified by calculation through a magnetic simulation using the finite element method as described above. As one example, FIG. 9 illustrates a calculation result. A horizontal axis represents a curvature radius of the concave surface. A unit of the curvature radius is mm. In this case, a width of each of the protruding portions is fixed to 8 mm, and a distance between the protruding portions is fixed to 8 mm. This is because the magnetic sensor has a square of sides 8 mm, and a size in which the magnetic sensor is insertable is adopted. In this case, curvatures of about 5.5 mm are optimal for the concave surfaces as the opposing surfaces of the protruding portions in order to maximize a magnetic flux density, and at this time, it can be seen that the magnetic flux density is increased to about 4%. Similarly, with use of the finite element method, it is possible to examine the concentrating structure that can increase the magnetic flux density.

One example of a feature of the concentrating structure that is applicable to the present embodiment will be described below. FIG. 10 illustrates a connection portion between the protruding portions and the band portion. It is desirable to connect the band portion with an appropriate curvature. This is because connection at an acute angle may cause disturbance in the flow of the magnetic flux. To cause the magnetic flux to smoothly flow as much as possible, the band portion has the curvature. As one example, a space of 8 mm is ensured with respect to the protruding portions with the lengths (protrusion lengths) of 10 mm, and the curvature may be set to about 4 mm. The curvature is not limited to the example as described above, but a curvature radius of about 10 mm is needed and it is better to ensure a curvature radius of at least a half of the space.

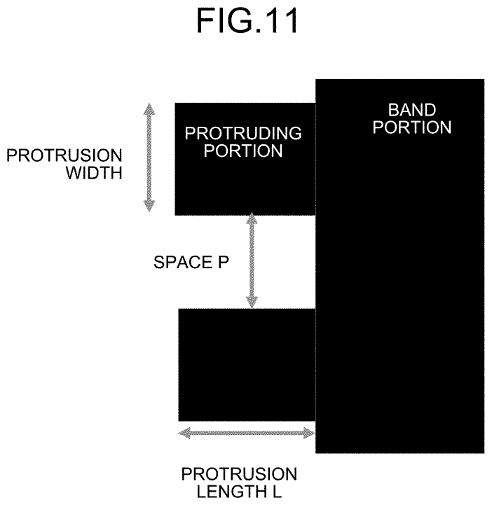

FIG. 11 illustrates the space and the protrusion lengths. A protrusion width is set to 8 mm that is the same size as the magnetic sensor. If the space does not have the equivalent width, it is difficult to fully concentrate the magnetic flux, and the magnetic flux flows to a portion where the magnetic sensor is not present. Further, each of the magnetic sensors has an internal coil, and therefore, crosstalk occurs. Therefore, if the space is not provided, the crosstalk occurs and data disturbance occurs. As one example, the space is set to 8 mm. In this case, it is desirable that the protrusion lengths are longer than the space. The protrusion lengths may be set to 10 mm, for example.

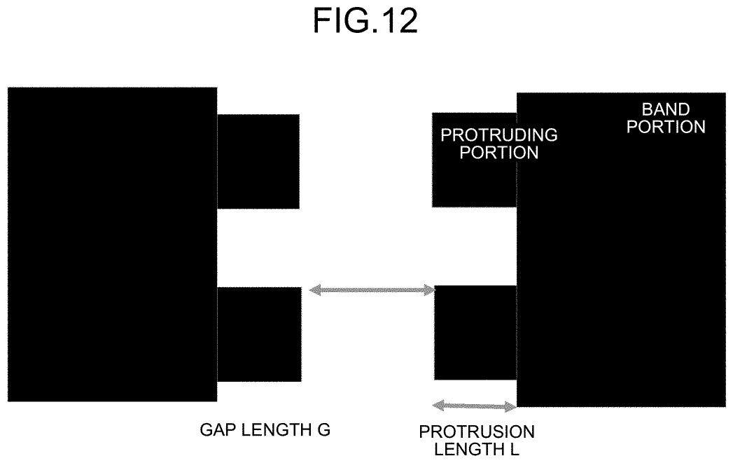

FIG. 12 illustrates an interval (gap) between the opposing protruding portions. A gap length is determined depending on the size of the magnetic sensor. As one example, if the magnetic sensor of 8 mm is used, it is possible to set the gap length to 8 mm. In this case, it is necessary to set the larger protrusion lengths than the gap length. If the protrusion lengths are smaller than the gap length, performance to concentrate the magnetic flux is reduced. As one example, the protrusion lengths may be set to 10 mm.

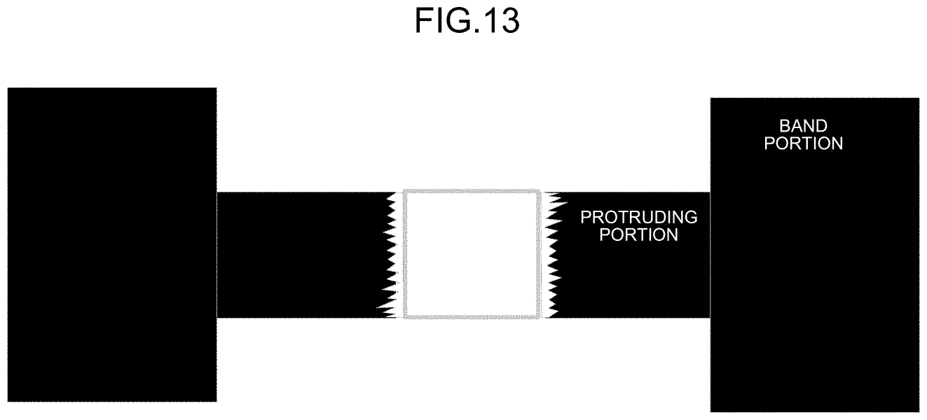

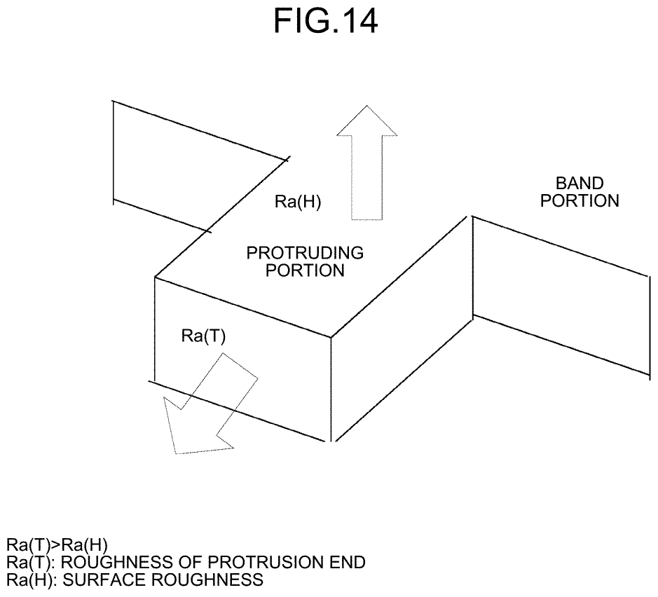

A protrusion end portion is a portion at which the magnetic flux is discharged from the inside of the ferromagnetic body with magnetic permeability of several hundred thousand to air or the magnetic sensor with magnetic permeability of about one. At an interface, reflection occurs and a phenomenon in which the magnetic flux is not discharged from a portion with high magnetic permeability may occur. The reflection depends on an angle of incidence on the interface. Therefore, as illustrated in FIG. 13, unevenness is intentionally provided on the protruding portions. With this configuration, the magnetic flux that is incident on the interface in an inclined manner increases, so that transmittance increases. In contrast, a surface portion in which the magnetic flux needs to be confined with high magnetic permeability as much as possible is formed in a flat manner. FIG. 14 illustrates control of unevenness in the end portion and the surface portion to use the phenomenon as described above. It is desirable to meet a condition of Ra(T)>Ra(H), where Ra(T) represents roughness of the protrusion end and Ra(H) represents the surface roughness.

It is preferable to use a material with high magnetic permeability as a material of the concentrating structure. Permalloy is one of materials that are generally used. The permalloy is composed of an alloy of Cu, Mo, Ni, Fe, or the like, and has maximum magnetic permeability of more than ten hundred. As one example, an amorphous metallic foil may be adopted. Examples of the amorphous metallic foil include “FINEMET (trademark)” that is a nanocrystalline Fe-based soft magnetic material. The amorphous metallic foil is preferable as compared to the permalloy because the amorphous metallic foil is light in weight and has good flexibility.

FIG. 15 is a diagram illustrating an example of a sensor configuration of a single unit of the first embodiment. In the present embodiment, the single detection sensor 3 and the single reference sensor 4 constitute a single unit. The protruding portions 6 are magnetically connected to the detection sensor 3. It is designed that a gap between the ferromagnetic bodies of the protruding portions 6 and a surface of the detection sensor 3 is only 100 micrometers (μm) or less. The protruding portions 6 and the detection sensor 3 are magnetically connectable only when they are arranged within at least 1 mm. In contrast, the reference sensor 4 is separated from the protruding portions 6 by about 10 mm, and no magnetic connection is established between them.

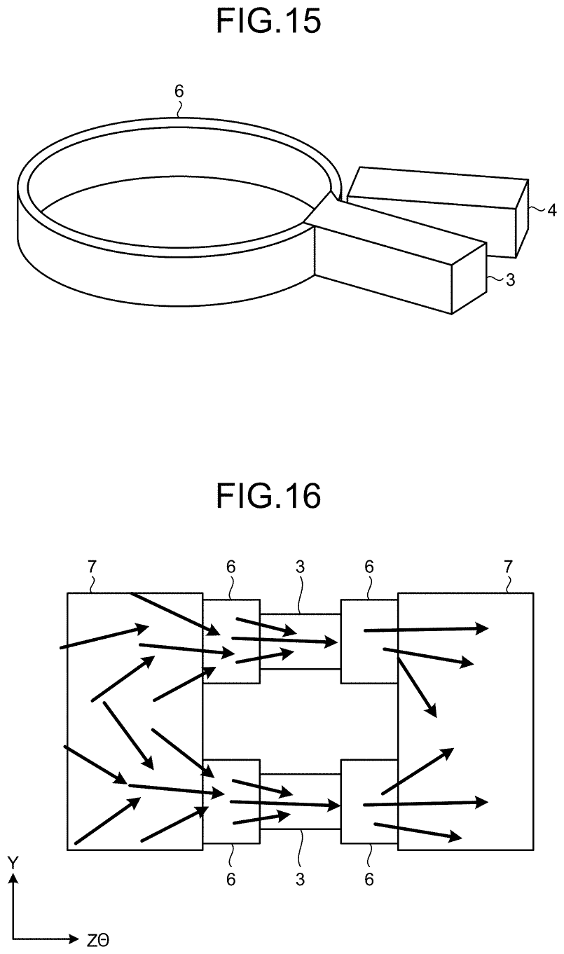

FIG. 16 is a diagram illustrating, by image, functions of the concentrating structure of the first embodiment. Arrows represent the magnetic flux and directions of the magnetic flux are represented by directions of the arrows. The magnetic flux preferentially propagates in a portion with high magnetic permeability. Therefore, when the magnetic flux generated from a living body passes through the band portion 7, the magnetic flux propagates while changing the orientation so as to be confined in the band portion 7. The band portion 7 covers the entire cervical spine, so that almost all magnetic flux is captured by the band portion 7. The magnetic flux propagates through the band portion 7 and reaches the protruding portions 6 that are magnetically connected to the band portion 7. End portions of the protruding portions 6 have small areas, and the magnetic flux is concentrated in the end portions. The concentrated magnetic flux is discharged from the protruding portions 6 to air. In this case, because the opposing protruding portions 6 are present, the magnetic flux preferentially propagates toward the opposing protruding portions. At this time, the magnetic flux passes through the detection sensor 3 that is arranged in an opposing position. The magnetic flux that passes through the detection sensor is twice or three times larger than that in a case in which the concentrating structure is not provided. This can be confirmed by calculation of intensity of a magnetic flux distribution through a simulation using the finite element method, for example.

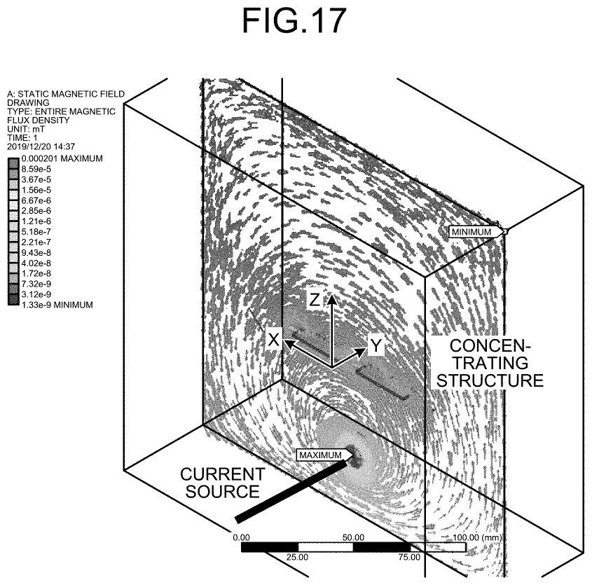

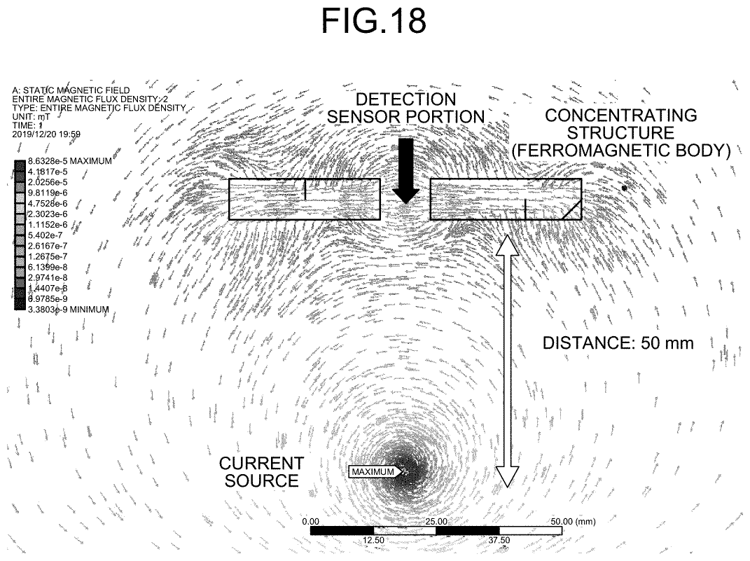

FIG. 17 and FIG. 18 are diagrams for explaining an example of a simulation (examples of a calculation model) of the concentrating structure of the first embodiment using the finite element method. In the example illustrated in FIG. 17 and FIG. 18, the concentrating structure is arranged at a position separated by about 50 mm from a current source. 50 mm is an imaginary distance from a position at which a nerve cell of the cervical spine is present to a skin surface. Two plates that mimic the protruding portions 6 of the concentrating structure are arranged with an interval of 8 mm on the skin surface. In the example illustrated in FIG. 17 and FIG. 18, it is assumed that the detection sensor 3 is arranged at the position of the interval of 8 mm.

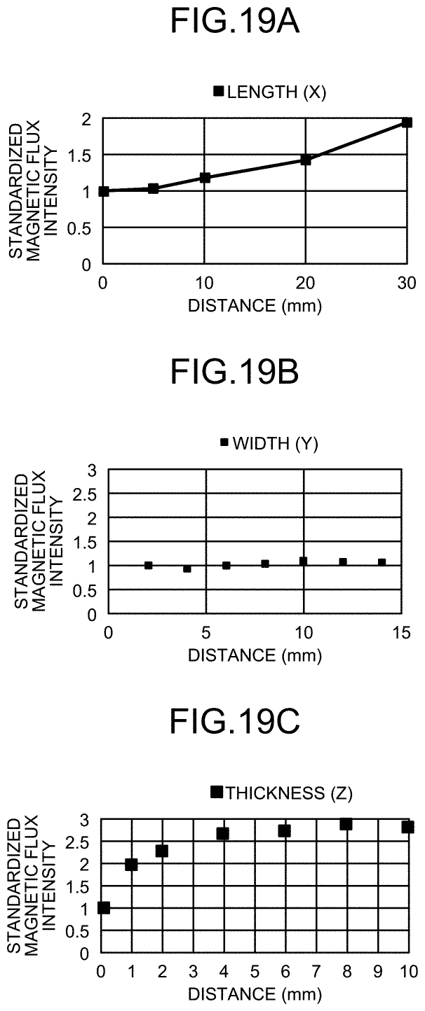

Here, FIG. 19A to FIG. 19C illustrate results of a magnetic flux density at the position of the detection sensor 3 when lengths (X direction), widths (Y direction), and thicknesses (Z direction) of the two plates in the coordinate system illustrated in FIG. 17 are changed.

FIG. 19A is a diagram illustrating the magnetic flux density when the lengths (X direction) are changed. FIG. 19B is a diagram illustrating the magnetic flux density when the widths (Y direction) are changed. FIG. 19C is a diagram illustrating the magnetic flux density when the thicknesses (Z direction) are changed. As illustrated in FIG. 19B, it can be seen that the magnetic flux density is not largely changed with respect to the widths (Y direction). It is assumed, by analogy, that if the widths are increased, the concentrated magnetic flux is spread proportionally. If the widths are increased, the magnetic flux that can be captured is increased, but the magnetic flux is also proportionally spread even in the portion of the detection sensor 3. In other words, it is desirable to set the widths to approximately the same as a sensitive surface (area in which the element is present) of the detection sensor 3.

In the present embodiment, the area of the detection sensor 3 is 8 mm×8 mm as will be described later, so that the widths are set to 8 mm. The widths correspond to the protruding portions 6 of the concentrating structure. It is desirable to widen the band portion 7 connected to the protruding portions 6 as much as possible. This is because the amount of magnetic flux captured by the magnetic body increases with an increase in the area. This is indicated even by a calculation result of dependency on the lengths as will be described later. In the present embodiment, the band portion 7 and the adjacently arranged detection sensor 3 are bonded in an overlapping manner to widen the band portion 7 as much as possible, which is characteristic. With this configuration, it is possible to concentrate maximum magnetic flux.

By increasing the thicknesses (Z direction), it is possible to increase the density of the magnetic flux concentrated on the detection sensors 3, and it is possible to achieve strength that is increased by about three times. However, the thicknesses (Z direction) are saturated at about 4 mm, and the effect is not changed even if the thicknesses are further increased. The same applies to almost all ferromagnetic bodies that are generally used, although it depends on the magnetic permeability. The ferromagnetic bodies, such as the permalloy, are heavy in weight, and therefore, it is possible to reduce a burden on the subject 100 by reducing the thicknesses as much as possible when the ferromagnetic bodies are to be mounted on the head portion.

Namely, it can be seen that the concentrating structure with the thickness of 4 mm is preferable because it is possible to increase the effect of the concentrating structure and reduce a burden on the subject 100. Therefore, in the first embodiment, the band portion 7 of the concentrating structure is realized by a plate-shaped magnetic body that covers the entire surface of a measurement portion of the subject 100 and that has a thickness of 4 mm or less. With this configuration, it is possible to realize light weight and easily cover the measurement portion of the subject 100. Further, by covering the entire surface of the measurement portion of the subject 100, it is possible to effectively concentrate biological magnetism that is generated from the measurement portion of the subject 100. It is possible to increase a biomagnetic signal that is input to the detection sensor 3, so that it is possible to improve apparent sensitivity.

It can be seen that, by setting the lengths (X direction) to about 30 mm, the magnetic flux density is increased by about twice. It is preferable to increase the lengths as much as possible if the adjacent detection sensor 3 is not provided. Increase in the concentrating efficiency with an increase in the lengths indicates that the concentrating efficiency increases with an increase in the area of the band portion 7. This indicates the same fact that a wide width of the band portion 7 is preferable as described above. The simulation calculation described here proofs that the present embodiment in which the band portion 7 is provided can largely improve the concentrating efficiency as compared to a conventional example in which the band portion 7 is not provided.

Detection Sensors

In the present embodiment, the MR sensors are adopted as the detection sensors 3 (magnetic sensors), but MI sensors, OPMs, or SQUIDs may be similarly applicable. The MR sensor has a cubic shape with a size of 8 mm×8 mm×70 mm. Wire is arranged on one end of the MR sensor in the longitudinal direction. The MR sensor is connected to the protruding portions 6 of the concentrating structure. A surface of the MR sensor is made of resin, and the surface is smoothed. Fixing slots having approximately the same shapes (8 mm×8 mm) and having depths of about 40 mm are formed on the protruding portions 6 of the concentrating structure such that the MR sensor with good slidability can be detachably attached. When the MR sensor is inserted in the slots, the protruding portions 6 of the concentrating structure and the MR sensor come close to each other with a clearance of about several tens μm, so that a magnetic connection can be established.

Reference Sensors

As the reference sensors 4, the same type of MR sensors as the MR sensors adopted as the detection sensors 3 as described above are selected. The MR sensors whose entire characteristics including shapes, sensitivity, and the like are most similar to those of the detection sensors are adopted. With this configuration, when the same magnetic flux enters the detection sensor 3 and the reference sensor 4, a difference is zero. If the difference is not zero, performance as the gradiometer is reduced. To detect extremely small magnetic flux as in the present embodiment, the reference sensors 4 need to have the same level of precision as that of the detection sensors 3.

As illustrated in FIG. 15, the reference sensor 4 is arranged so as to be adjacent to the detection sensor 3 with an interval of about 20 mm by which the crosstalk does not become a problem. The detection sensor 3 is magnetically connected to the protruding portions 6 of the concentrating structure, but the reference sensor 4 is arranged so as to be magnetically insulated as much as possible. In the present embodiment, the ferromagnetic body with a plate thickness of about 4 mm is arranged as the concentrating structure between a signal source of a living body and the reference sensor 4, so that magnetic insulation is realized. In particular, the reference sensor 4 is separated from an end portion (in this example, the protruding portion 6) of the magnetic body as much as possible, and is arranged near a smooth flat surface. With this configuration, the magnetic field of the biological signal does not enter the reference sensor 4 at all. With respect to external noise, because the detection sensor 3 and the reference sensor 4 are arranged adjacent to each other, approximately the same signal is input and a biological signal is not input, so that it is possible to realize a high-accuracy gradiometer.

Further, by creating a condition for inverse problem estimation to be described later, it is possible to remove, through calculation, uniform external magnetic field noise that is distorted by the concentrating structure.

Configuration of Magnetic Measurement System

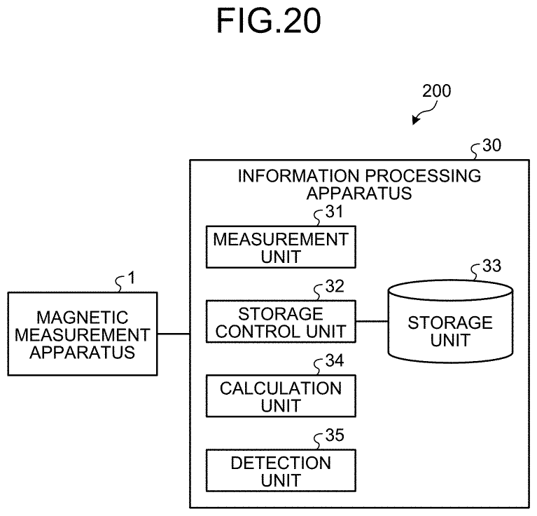

FIG. 20 is a diagram illustrating an example of a configuration of a magnetic measurement system 200 of the first embodiment. The magnetic measurement system 200 of the first embodiment includes the magnetic measurement apparatus 1 (the magnetospinography in the first embodiment) and an information processing apparatus 30.

The magnetic measurement apparatus 1 and the information processing apparatus 30 are communicably connected to each other in a wired or wireless manner. The information processing apparatus 30 receives a biomagnetic signal measured by the magnetic measurement apparatus 1 from the magnetic measurement apparatus 1, for example.

The information processing apparatus 30 of the first embodiment includes a measurement unit 31, a storage control unit 32, a storage unit 33, a calculation unit 34, and a detection unit 35.

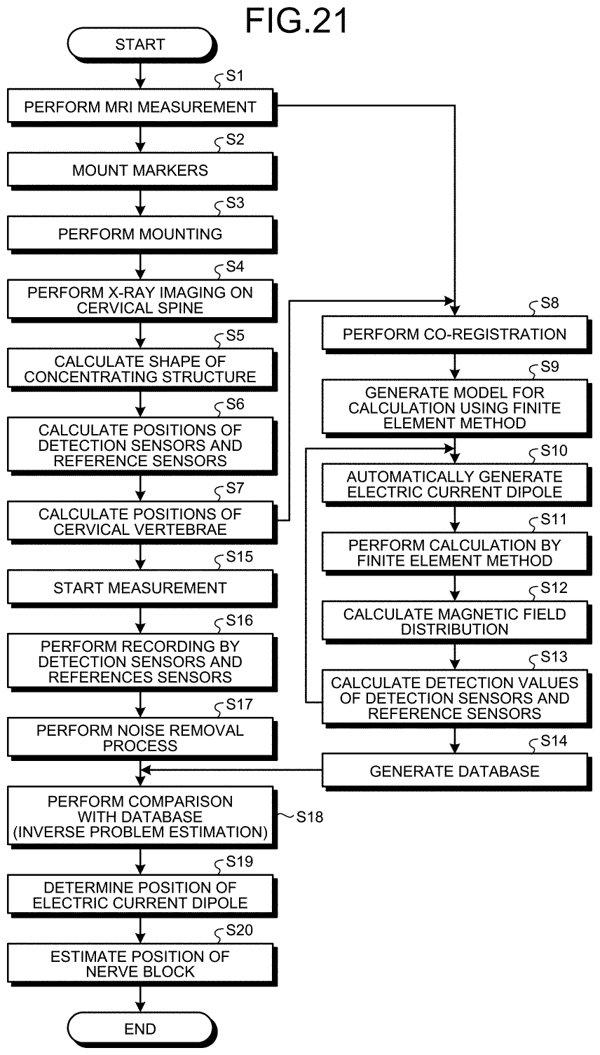

The measurement unit 31 performs a measurement process. For example, the measurement unit 31 measures, from an X-ray imaging result or the like of a subject (the subject 100 in the first embodiment), a three-dimensional shape of the concentrating structure (the protruding portions 6 and the band portion 7) and a three-dimensional shape of the subject (three-dimensional data of an MRI image of the subject 100 in the first embodiment). Further, for example, the measurement unit 31 measures a magnetic field distribution by the finite element method, from the three-dimensional shape of the concentrating structure, the three-dimensional shape of the subject, magnetic permeability of the three-dimensional shape of the subject, and magnetic permeability of the three-dimensional shape of the concentrating structure. Meanwhile, details of a process performed by the measurement unit 31 will be described later with reference to FIG. 21.

The storage control unit 32 performs control of storing data in the storage unit 33. For example, the storage control unit 32 stores the biomagnetic signal measured by the magnetic measurement apparatus 1 in the storage unit 33.

The storage unit 33 stores therein data. For example, the storage unit 33 stores therein the biomagnetic signal measured by the magnetic measurement apparatus 1. Further, for example, the storage unit 33 stores therein a database of computer-aided design (CAD) information or the like on the three-dimensional shape of the concentrating structure (the protruding portions 6 and the band portion 7).

The calculation unit 34 performs inverse problem estimation by using the magnetic field distribution and a magnetic signal (the biomagnetic signal measured by the magnetic measurement apparatus 1 in the first embodiment), and calculates a current distribution of the subject. Meanwhile, details of a process performed by the calculation unit 34 will be described later with reference to FIG. 21.

The detection unit 35 detects a position at which neurotransmission is blocked, from the current distribution that is calculated through the inverse problem estimation.

Meanwhile, details of a process performed by the detection unit 35 will be described later with reference to FIG. 21.

Example of Measurement Method

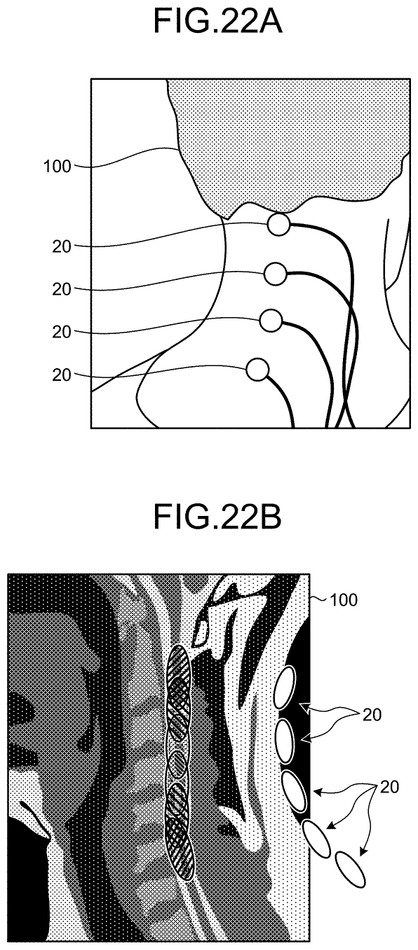

FIG. 21 is a flowchart illustrating an example of a measurement method of the first embodiment. First, MRI measurement on the subject 100 is performed and an MRI image is acquired (Step S1). Subsequently, markers 20 as illustrated in FIG. 22A and FIG. 22B are mounted on the subject 100 (Step S2).

FIG. 22A and FIG. 22B are diagrams illustrating a mounting example of the markers 20 of the first embodiment. When the markers 20 are to be mounted on the subject 100, the subject 100 is requested to sit on a stable chair and protective equipment is set to stabilize a posture. As the way of sitting, the subject leans on a backrest, and a support is needed on the head portion to prevent movement during measurement. The backrest is extended and adjusted to receive the weight of the subject such that the subject is stabilized. Depending on the state of the subject 100, it may be possible to adopt a form in which the weight of the subject is supported by a breast with face down, or a form in which the subject is lying down on a bed with face up. The markers 20 are mounted in the stable state as described above.

The markers 20 include coils capable of generating alternating current magnetic flux of about 1 kHz, are made of a metal material, and have certain shapes that allow confirmation of positions such that the positions can be confirmed at the time of X-ray imaging. The markers 20 are mounted at C2 to C7 (positions of the cervical vertebrae) by determining feature points of the cervical vertebrae on palpation. As illustrated in FIG. 22A, it is sufficient to mount the markers 20 at about four positions, and may be intentionally deviated by about 20 mm in a horizontal direction from the positions of the cervical vertebrae. Measurement is performed while the markers 20 are mounted, so that an influence of noise or the like may occur. To reduce the influence, the markers 20 are intentionally deviated such that the markers 20 are not arranged between nerves and the detection sensors 3.

Referring back to FIG. 21, in the state as illustrated in FIG. 22A and FIG. 22B, the concentrating structure (the protruding portions 6 and the band portion 7), the detection sensors 3, and the reference sensors 4 are mounted on the subject 100 (Step S3). The method of fixing the concentrating structure (the protruding portions 6 and the band portion 7) the detection sensors 3, and the reference sensors 4 to the subject 100 is described above, and therefore, explanation thereof will be omitted.

Subsequently, an X-ray imaging device performs X-ray imaging on the subject 100 in this state, and acquires an X-ray image (Step S4). The X-ray imaging is performed in two directions, that is, in a front direction and a lateral direction. Then, the measurement unit 31 measures, from the X-ray image, the positions and orientations of the detection sensors 3, the positions and orientations of the reference sensors 4, the shape of the concentrating structure, the positions of the markers 20, and the positions of the cervical vertebrae C1 to C7, and calculates data indicating the positions, the orientations, and the shapes (Step S5 to Step S7).

Specifically, first, the concentrating structure is read as having a cylindrical outer shape, and a diameter and a position in the length direction are recorded in the storage unit 33. The concentrating structure having the cylindrical outer shape includes the band portion 7 and the protruding portions 6; therefore, the storage unit 33 records therein, in advance, CAD information on the three-dimensional shape of the concentrating structure (the protruding portions 6 and the band portion 7) such that the shapes can also be recorded.

Subsequently, the measurement unit 31 measures, from the X-ray image acquired at Step S4, the coordinates of apical end positions of the detection sensors 3 and the reference sensors 4 and three-dimensional rotated coordinates indicating the orientations of the detection sensors 3 and the reference sensors 4. The storage unit 33 stores therein the coordinates of the apical end positions and the three-dimensional rotated coordinates.

The detection sensors 3 and the reference sensors 4 incorporate therein the plurality of (about four) markers 20 that clearly appear when X-ray imaging is performed. In the storage unit 33, three-dimensional CAD information on the detection sensors 3 and the reference sensors 4 is recorded in advance such that six-order data representing the positions and the rotation of the detection sensors 3 and the reference sensors 4 can easily be calculated from the positions of the markers 20.

Finally, the measurement unit 31 measures the positions of the cervical vertebrae C1 to C7 from the X-ray image that is acquired at Step S4, and calculates the three-dimensional coordinates of the positions of the cervical vertebrae. The shapes of the cervical vertebrae have individual differences, and therefore, it is not possible to use CAD data prepared in advance unlike the detection sensors 3 and the concentrating structure as described above. Therefore, in the present embodiment, the measurement unit 31 performs mechanical learning on a large number of X-ray photographs. Accordingly, even for the position of each of the cervical vertebrae that have large individual differences, the measurement unit 31 is able to easily calculate the three-dimensional coordinates of the positions of the cervical vertebrae. The measurement unit 31 calculates positions of the centers of gravity of the cervical vertebrae as the three-dimensional coordinates indicating the positions of the cervical vertebrae.

Subsequently, the measurement unit 31 performs co-registration (position synchronization) of the MRI image that is acquired through the MRI measurement at Step S1 and data indicating the positions, the orientations, and the shapes calculated at Step S5 to Step S7 (Step S8).

Subsequently, the measurement unit 31 generates a model for calculation using the finite element method (Step S9). The MRI image is the three-dimensional data, and therefore, the model for the finite element method is also a three-dimensional model. Meanwhile, if the MRI image is not provided, it may be possible to use an image that is adjusted from an MRI image of a standard cervical spine with reference to an X-ray image.

Conventionally, it is assumed that the magnetic permeability of the subject is substantially equal to that of air, and direct problem calculation has been performed through analytical calculation based on the Biot-Savart's law. In the conventional method, if a structural object with high magnetic permeability is present as in the present embodiment, calculation is largely deviated, and an error in the inverse problem estimation increases. To cope with this, by measuring the shape of the concentrating structure with high magnetic permeability in advance and calculating the magnetic field distribution by the finite element method, it is possible to implement accurate direct problem calculation. Consequently, it is possible to accurately perform inverse problem estimation. Thus, it is possible to provide a biomagnetic apparatus capable of perform measurement with high accuracy.

The measurement unit 31 may perform an electromagnetic field simulation of the finite element method by using a tool that is commercially available. This method will be described below. First, the measurement unit 31 cuts the three-dimensional data of the MRI image of the subject 100 into a mesh shape and generates a voxel for performing the finite element method. A size of the voxel is adjusted in accordance with a calculator (the information processing apparatus 30) because the size affects a calculation volume.

In the present embodiment, the voxel of a square of sides 1 mm is adopted, and a calculation region as a cube of sides 200 mm is adopted to fully cover the cervical vertebrae C1 to C7. The measurement unit 31 performs classification, from the shape data, into bones, nerves, muscles, fat, the concentrating structure (the ferromagnetic body), the detection sensors 3, the reference sensors 4, and the fixing jig 2, and sets magnetic permeability and conductivity of each of the classified objects. Thus, the model for the electromagnetic field simulation is completed.

Subsequently, the measurement unit 31 automatically generates an electric current dipole (Step S10). The electric current dipole has a certain shape including a pair of current pieces. The measurement unit 31 arranges the electric current dipole at a position of a nerve, and performs a simulation of a magnetic field distribution by the finite element method (Step S11 and Step S12).

Accordingly, it is possible to recognize how much magnetic flux is generated at the positions of the detection sensors 3 and the positions of the reference sensor 4 (Step S13). The magnetic field is a vector. Further, intensity depends on intensity of the electric current dipole and is difficult to be estimated; therefore, information on the vector or a relative value is adopted as useful information.

Returning to Step S10, the electric current dipole is moved along the nerve. A movement distance is about 1 mm although it depends on the size of the mesh. The calculation region is 200 mm, and therefore, the calculation is repeated 200 times. Through the repetition of the calculation 200 times, a database in which data corresponding to the detection sensors 3 at 10 positions with respect to the electric current dipoles at 200 positions are stored as three-dimensional vector magnetic fields is generated (Step S14). Meanwhile, if the positions of the detection sensors 3 are not determined, the measurement unit 31 may generate a database of the magnetic field vectors at all of the 200 positions of the electric current dipole, at all of the positions in the calculation region of the cube of sides 200 mm.

In contrast, when the biomagnetic signal of the subject 100 is to be measured, an electrode that inputs a stimulus is mounted on a cubital region or a wrist, and the magnetic measurement apparatus 1 starts measurement in a state in which the stimulus is input (Step S15). In the measurement, a stimulus at 10 Hz is accumulated 5000 times. A measurement time is about 10 minutes. Each of the detection sensors 3 and the reference sensors 4 records data obtained in 10 minutes, at a sampling rate of 10 kHz (Step S16). The storage control unit 32 receives the data obtained in the 10 minutes from the magnetic measurement apparatus 1 and stores the data as a measurement result in the storage unit 33.

Subsequently, the measurement unit 31 performs a noise removal process on the measurement result (Step S17). Specifically, the measurement unit 31 first performs the noise removal process on the measurement result by using a low-pass filter and a high-pass filter, and further performs a process of calculating a difference between data recorded by the detection sensors 3 and data recorded by the reference sensors 4 (subtraction process for disturbance noise) (Step S17). Finally, the measurement unit 31 averages the accumulated 5000 pieces of data, and the storage control unit 32 stores the averaged data in the storage unit 33. Consequently, data of all of the detection sensors 3 are acquired as a timeline of 100 milliseconds (msec).

Subsequently, the calculation unit 34 compares the timeline of 100 msec (100 msec (100 times) at an interval of a timeline of 1 msec) and the database generated at Step S14, and performs the inverse problem estimation for calculating the position of the electric current dipole (Step S18). Accordingly, it can be clearly seen that the electric current dipole that has first received the stimulus and moved to the cervical vertebra after latency appears at C7 after a lapse of dozens of seconds, and moves upward to C6 and C5. In 100 repetitions of calculation (100 msec at an interval of 1 msec), it is possible to determine the position of the electric current dipole at each calculation (Step S19).

Finally, the detection unit 35 detects a position at which the electric current dipole disappears (Step S20). The position at which the electric current dipole disappears is a position at which nerve block has occurred.

Through the process in the flowchart as described above, it is possible to estimate the position of the nerve block.

Thus, as described above, the magnetic measurement apparatus 1 of the first embodiment includes a plurality of magnetic sensors (the detection sensors 3) that are arranged in a first direction (the Y direction in FIG. 6), and a concentrating structure. The concentrating structure includes the band portion 7 that covers a measurement portion of a subject (the subject 100 in the first embodiment), and includes the plurality of protruding portions 6. The plurality of protruding portions 6 are configured such that pairs of the two protruding portions 6 facing each other in a second direction (the Zθ direction in FIG. 6) are arranged in the first direction. Each of the magnetic sensors is arranged between the two protruding portions 6 of each of the pairs.

With this configuration, according to the magnetic measurement apparatus 1 of the first embodiment, it is possible to improve inadequate sensitivity of the magnetic sensors (the detection sensors 3) that are available under geomagnetism and at ordinary temperature, and capture magnetic signals with high accuracy. Specifically, with a shape in which a structure for covering the entire region of the subject (the measurement portion of the subject 100 in the first embodiment) and concentrating magnetic flux by the band portion 7 and a structure for concentrating the magnetic flux to the detection sensors 3 are simultaneously provided, it is possible to improve inadequate sensitivity of the detection sensors 3. Further, by covering the cervical spine with a plate of the magnetic body (with a length of 30 mm or more and a thickness of 4 mm or more), it is possible to capture biomagnetic signals generated from the cervical spine into the concentrating structure (the protruding portions 6 and the band portion 7) without any missing part. Furthermore, by providing the protruding portions 6 in a gap of the magnetic body, it is possible to concentrate the magnetic flux in the detection sensors 3.

### Second Embodiment

A second embodiment will be described below. In the description of the second embodiment, the same explanation as those of the first embodiment is omitted, and differences from the first embodiment will be described. In the second embodiment, a magnetoencephalography including a concentrating structure for capturing a brain signal will be described.

A large number of brain diseases that are worth to be captured by the magnetoencephalography exist, and by objectively capturing brain signals, it is possible to improve accuracy of intervention treatment, such as medicine or rehabilitation. For example, if sensory stimuli are not transmitted to the brain due to spinal cord injury or the like, it is expected to perform treatment by regenerative medicine using iPs cells or the like.

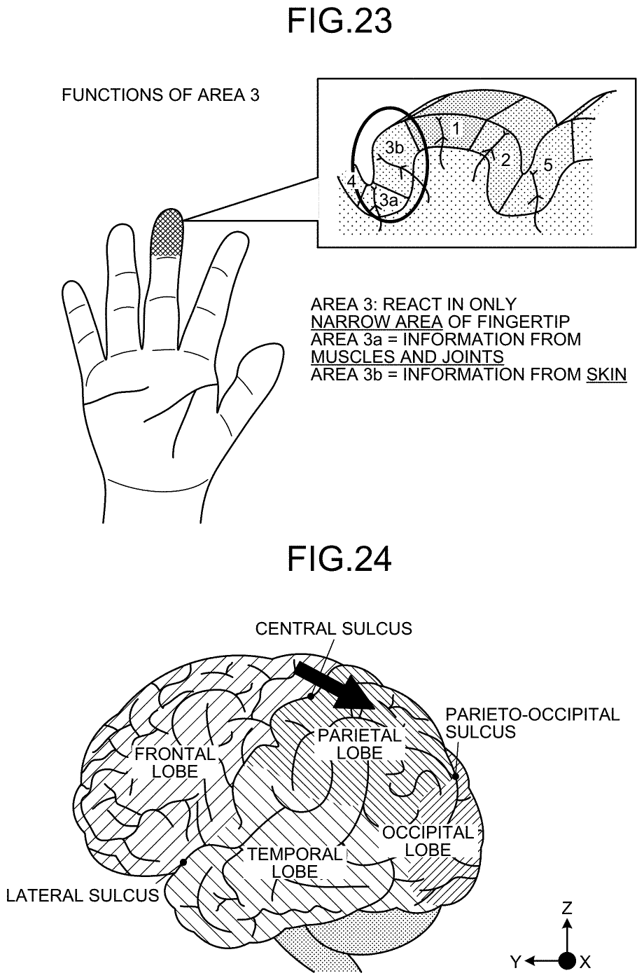

FIG. 23 is a diagram for explaining brain functions and areas. FIG. 24 is a diagram for explaining a direction in which a dipole occurs. If an electrical stimulus is given to a finger of the subject 100, a signal occurs in Brodmann area 3a or the like (see FIG. 23) that is a brain sensory area. The occurrence position is located in a central sulcus in FIG. 24. It is possible to quantitatively evaluate a recovery state of brain injury by detecting a signal of the magnetoencephalography.

In the present embodiment, a magnetoencephalography that is used to effectively perform rehabilitation on a person who has disturbance of motor function or the like due to a stroke or the like will be described. In recent years, neurorehabilitation or the like using an electroencephalography or a near infrared spectroscopy (NIRS) using near infrared archives high treatment results.

The magnetoencephalography has higher spatial resolution than that of the electroencephalography and a faster response speed than that of the NIRS, and therefore is expected to achieve higher effects than the modalities as described above. This is because when the subject 100 is to activate the brain function on his/her own will, it is possible to give a feedback on whether a signal is generated in an appropriate manner and clearly provide the feedback to the subject 100, so it is possible to guide to an appropriate brain activity at early stage. For example, when a person has an intention to move a finger, and if the person is a healthy person, a neuron fires as a neural activity at around a motor cortex (around the central sulcus corresponding to a parietal region) of the brain. If a stroke has occurred and peripheral brain functions are damaged, anon-appropriate positions, such as a frontal lobe and an occipital lobe, in a wide area fire when the finger is moved. If rehabilitation is processed and symptoms are improved, the brain activities are changed to local activities. One benchmark is to cause the brain function to work as a local activity. By providing a response to the subject 100 only when only the position of an accurate central sulcus fires at the time the subject 100 intends to move a finger, it is possible to cause the brain function to accurately operate. By giving the feedback, it is possible to improve a treatment effect.

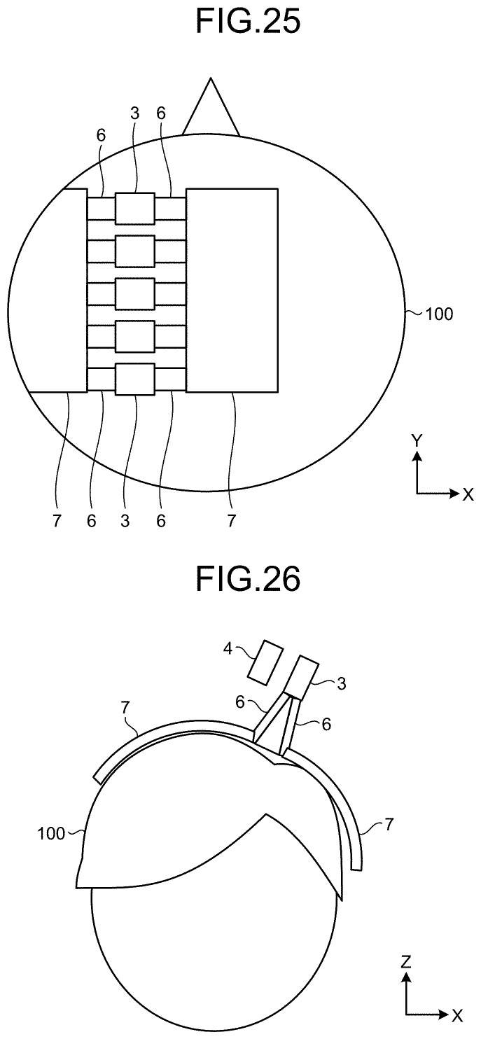

The central sulcus extends in a lateral direction in the parietal region, and serves as the X direction in FIG. 24. At this time, a dipole (indicated by an arrow) by which an electric current flows in the perpendicular direction (Y direction) is generated. This is determined by arrangement of nerve cells that are arranged perpendicular to a wall of the central sulcus. The concentrating structure that increases intensity of the signal and improves apparent sensitivity has a certain shape as illustrated in FIG. 25.

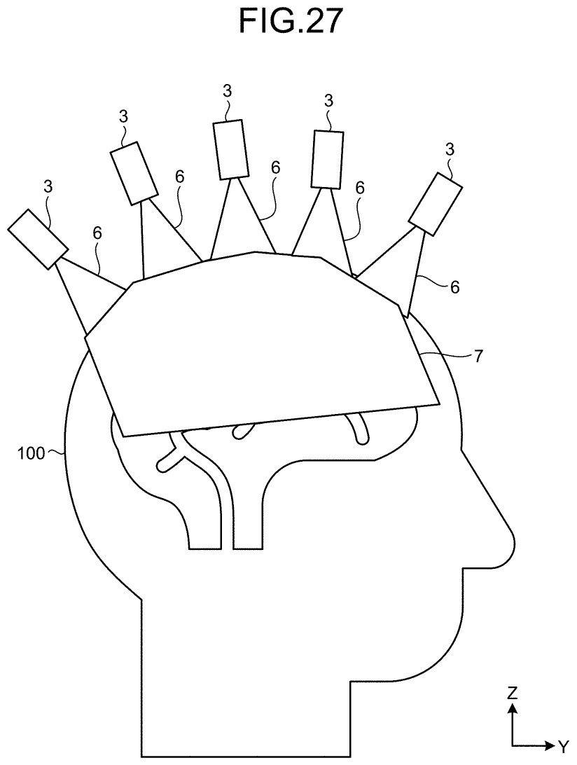

FIG. 25 to FIG. 27 are diagrams illustrating an example of arrangement of the concentrating structure (the protruding portions 6 and the band portions 7) of the second embodiment. The arrow illustrated in FIG. 24 indicates the dipole of the neural activity that has fired in the central sulcus. When the subject 100 faces in the Y direction, the detection sensors 3 are arranged in the Y direction. FIG. 26 illustrates the subject 100 when viewed from front. The concentrating structure of the second embodiment includes the protruding portions 6 and the band portions 7, and the second embodiment is characterized in that the protruding portions 6 stand in the Z direction. The reference sensors 4 are arranged adjacent to the detection sensors 3. FIG. 27 is a side view. In the example illustrated in FIG. 27, the five detection sensors 3 are arranged in the Y direction.

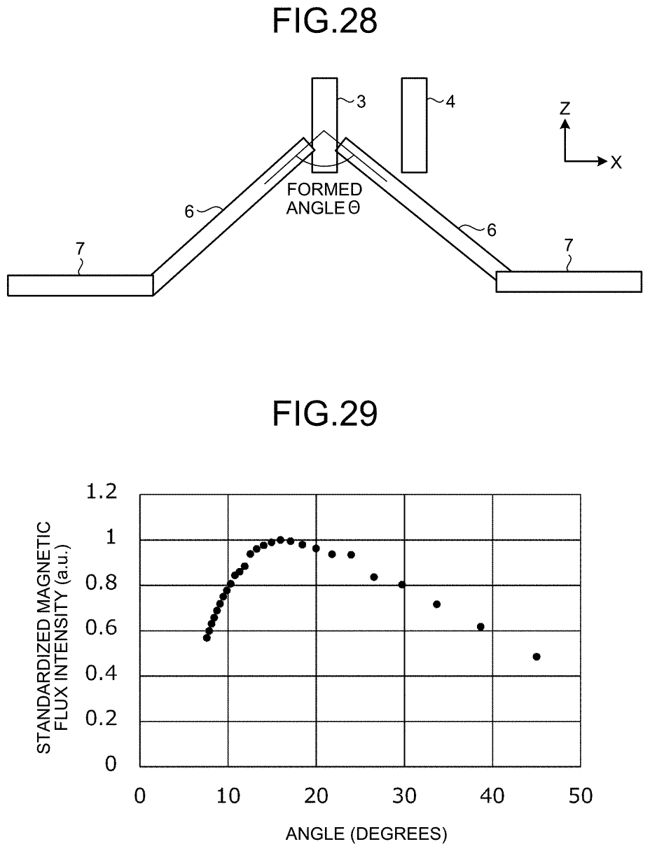

FIG. 28 is a diagram illustrating an angle θ formed by the two protruding portions 6 of the second embodiment. FIG. 29 is a diagram illustrating a relationship between the angle θ formed by the two protruding portions 6 of the second embodiment and magnetic flux intensity. In the example illustrated in FIG. 29, the relationship between the angle θ and the magnetic flux intensity generated by the detection sensors 3 is illustrated as a calculation result of an electromagnetic field distribution using the finite element method. A vertical axis in FIG. 29 represents the magnetic flux intensity. A horizontal axis in FIG. 29 represents a value of the angle θ.

If the concentrating structure is made of a plate of a magnetic body made of permalloy, the magnetic flux intensity gradually increases with a decrease in the angle θ. This indicates that the magnetic flux flows into the concentrating structure and the flow is concentrated. Further, if the angle θ is further reduced, the magnetic flux flows from the band portion 7 to the adjacent band portions 7, so that the magnetic flux is not discharged to the detection sensors 3. Therefore, there is an appropriate value of the angle θ. If the angle θ is equal to or larger than 180 degrees, the magnetic flux is not discharged to the detection sensors 3, and therefore, the angle θ needs to be smaller than 180 degrees. Further, the angle θ needs to be equal to or larger than 180 degrees to arrange the reference sensors 4 above the protruding portions 6, and interference occurs in other cases. Moreover, if the angle θ is equal to or smaller than 10 degrees, the magnetic flux flows from one of the band portions 7 to another one of the band portions 7, and therefore, it can be seen that the angle θ needs to be equal to or larger than 10 degrees. Therefore, in the present embodiment, the angle θ is set to 15 degrees. By setting the angle θ to be equal to or larger than 10 degrees and equal to or smaller than 20 degrees, it is possible to largely improve magnetic concentrating efficiency and further reduce a biomagnetic signal that is input to the reference sensors 4.

Example of Measurement Method

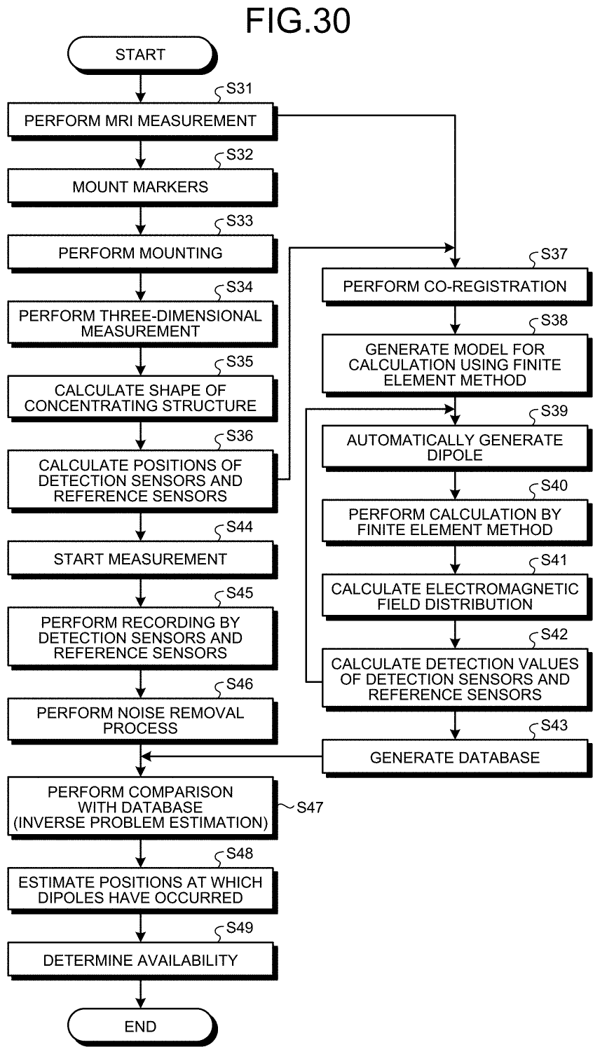

FIG. 30 is a flowchart illustrating an example of a measurement method of the second embodiment. First, MRI measurement on the subject 100 is performed and an MRI image is acquired (Step S31). In the second embodiment, the markers 20 to be detected in the MRI image are mounted on feature points, such as a glabella or a temple, of the subject 100, and the MRI image is acquired. In the MRI image, a brain structure that is an object of interest and the positions of the markers 20 are recorded. The MRI image is used to confirm a position of the central sulcus in which the dipole that is a motor action is generated.

Subsequently, the subject 100 is requested to sit on a stable chair and the markers 20 are mounted in the same manner at the same positions as mounted in the MRI in the stabile state (Step S32). The markers 20 include coils capable of generating alternating current magnetic flux of about 1 kHz. In this state, the concentrating structure, the detection sensors 3, and the reference sensor 4 are mounted (Step S33), and three-dimensional measurement is performed (Step S34). In the second embodiment, the three-dimensional measurement is performed by a stereo camera.

The measurement unit 31 automatically calculates the positions of the detection sensors 3 and the reference sensor 4, the shape of the concentrating structure, and the positions of the markers 20 from a result of the three-dimensional measurement performed at Step S34, and assigns three-dimensional coordinates (Step S35 and Step S36). In the storage unit 33, CAD information on the three-dimensional shape of the concentrating structure (the protruding portions 6 and the band portions 7) is recorded in advance. The measurement unit 31 identifies, with use of the CAD information, the shape of the concentrating structure from a result of the three-dimensional measurement performed at Step S34.

The measurement unit 31 measures the coordinates of the apical end positions of the detection sensors 3 and the reference sensors 4 and the three-dimensional rotated coordinates indicating orientations of the detection sensors 3 and the reference sensors 4 from the three-dimensional image. The storage unit 33 stores therein the coordinates of the apical end positions and the three-dimensional rotated coordinates.

The detection sensors 3 and the reference sensors 4 include, on surfaces thereof, the plurality of (about four) markers 20 that clearly appear when the three-dimensional measurement is performed. In the storage unit 33, three-dimensional CAD information on the detection sensors 3 and the reference sensors 4 is recorded in advance such that six-order data representing the positions and the rotation of the detection sensors 3 and the reference sensors 4 can easily be calculated from the positions of the markers 20.

Subsequently, the measurement unit 31 performs co-registration (position synchronization) of the positions of the markers 20, such as the glabella or the temple, in the MRI image of the inside of the head portion acquired through the MRI measurement at Step S1 and the positions of the markers 20 in the three-dimensional image (Step S37). Accordingly, it becomes possible to identify position-and-shape data of the detection sensors 3, position-and-shape data of the reference sensors 4, and position-and-shape data of the concentrating structure in the MRI image.

Subsequently, the measurement unit 31 generates a model for the finite element method from the MRI image (Step S38). The MRI image is the three-dimensional data, and therefore, the model for the finite element method is also a three-dimensional model. Meanwhile, if the MRI image of the subject 100 is not provided, it may be possible to use an image that is adjusted from an MRI image of a standard brain to generate the model for the finite element method.

The measurement unit 31 may perform an electromagnetic field simulation using the finite element method by using a tool that is commercially available. This method will be described below. First, the measurement unit 31 cuts the three-dimensional data of the MRI image of the subject 100 into a mesh shape and generates a voxel for performing the finite element method. A size of the voxel is adjusted in accordance with a calculator (the information processing apparatus 30) because the size affects a calculation volume.

In the present embodiment, the voxel of a square of sides 3 mm is adopted, and a calculation region as a cube of sides 200 mm is adopted to fully cover the head portion. The measurement unit 31 performs classification, from the shape data, into the brain shape, the concentrating structure (ferromagnetic body), the detection sensors 3, the reference sensors 4, and the fixing jig 2, and sets magnetic permeability and conductivity of each of the classified objects. Thus, the model for the electromagnetic field simulation is completed.

Subsequently, the measurement unit 31 arranges the dipole in the calculation region (Step S39). Then, the measurement unit 31 performs a simulation of the magnetic field distribution by the finite element method (Step S40 and Step S41). Accordingly, it is possible to recognize how much magnetic flux is generated at the positions of the detection sensors 3 and the positions of the reference sensor 4 (Step S42).

Returning to Step S39, the position of the dipole is moved. The measurement unit 31 performs a simulation of the magnetic field distribution at the moved position. The measurement unit 31 records magnetic flux intensity at the positions of the detection sensors 3 and the positions of the reference sensors 4. The measurement unit 31 performs the above-process on all of positions in a gray matter in the MRI image and generates a database (Step S43).

In contrast, after Step S36, the magnetic measurement apparatus 1 of the second embodiment (the magnetoencephalography in the second embodiment) starts measurement on the subject 100 (Step S44). In the second embodiment, when a biomagnetic signal of the subject 100 is to be measured, the subject 100 is requested to imagine a tapping task for a middle finger of a right hand at 1 Hz. An important task is to imagine a case in which a finger does not actually move, during a brain function treatment process. The tapping imagination is performed for 10 seconds, and then a rest state (listing task) is performed for the same 10 seconds as a reference. This set is repeated 10 times. A task of about three minutes in total is performed.

Each of the detection sensors 3 and the reference sensors 4 records data obtained in the three minutes, at a sampling rate of 10 kHz (Step S45). The storage control unit 32 receives the data obtained in the three minutes from the magnetic measurement apparatus 1 and stores the data as a measurement result in the storage unit 33.

Subsequently, the measurement unit 31 performs a noise removal process on the measurement result (Step S46). Specifically, the measurement unit 31 first performs the noise removal process on the measurement result by using a low-pass filter and a high-pass filter, and further performs a process of calculating a difference between data recorded by the detection sensors 3 and data recorded by the reference sensors 4 (subtraction process for disturbance noise) (Step S17). Finally, the measurement unit 31 accumulates data of the tapping and the listing each performed 10 times, where each of the tapping and the lifting is performed for 10 seconds each time. Then, the measurement unit 31 calculates a difference between data of a tapping task period and data of a listing period. Accordingly, it is possible to calculate detection values of the detection sensors 3 located at five positions.

In the second embodiment, it is intended to estimate a pattern in which dipoles simultaneously occur at a plurality of positions. The magnetic field distributions generated by the dipoles can be linearly added. There are countless probabilities of possible estimation on positions, intensity, the number, and locations of the dipoles from actual measurement results. A most probable pattern is calculated from the countless probabilities. There are countless patterns that may be adopted as solutions, as compared to the measurement results that are obtained through the processes from Step S44 to Step S46, so that it is impossible to uniquely identify a solution. This is generally regarded as an ill-posed problem, but the calculation unit 34 and the detection unit 35 estimate a probable answer as the inverse problem estimation (Step S47 to Step S49).

### Third Embodiment

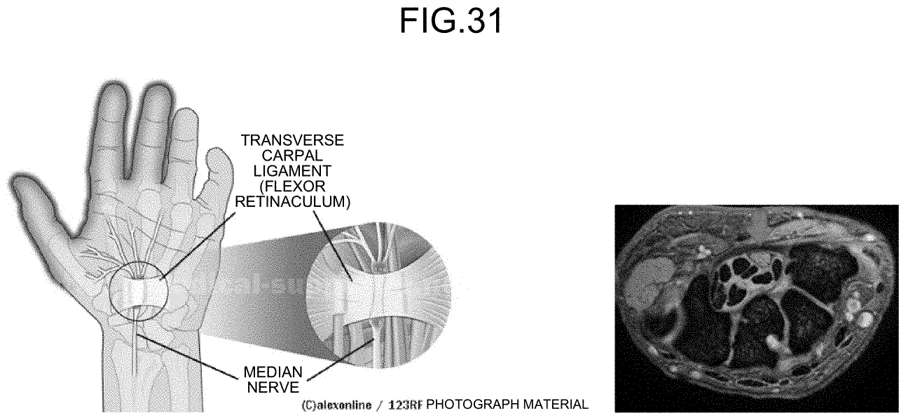

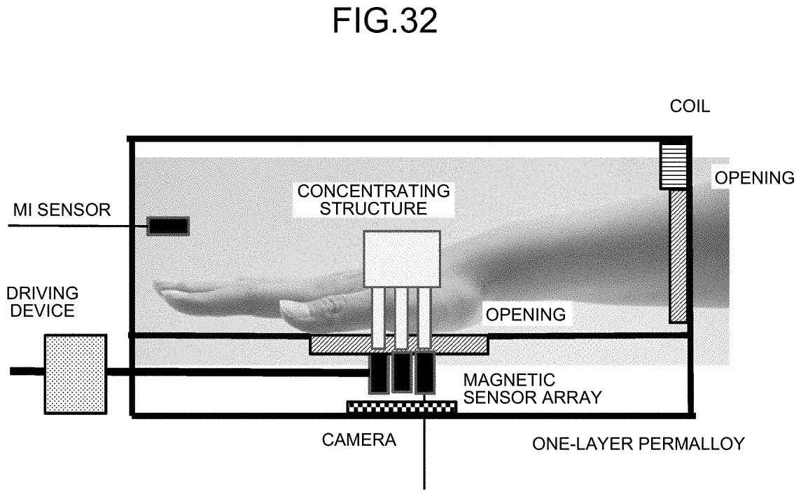

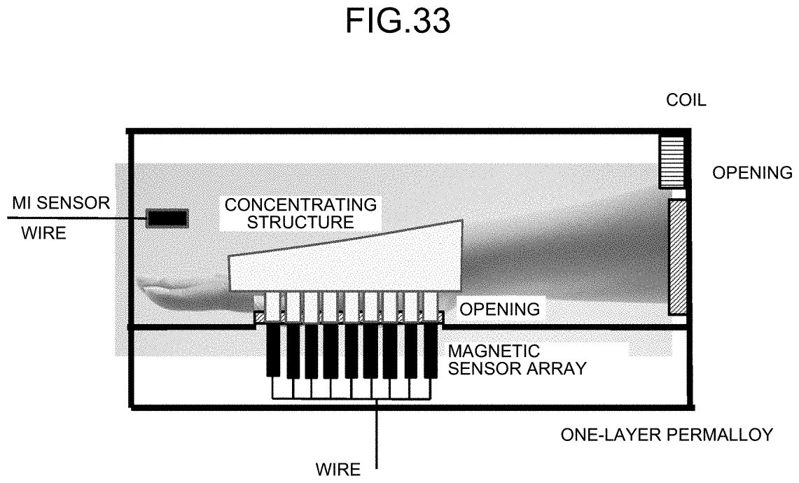

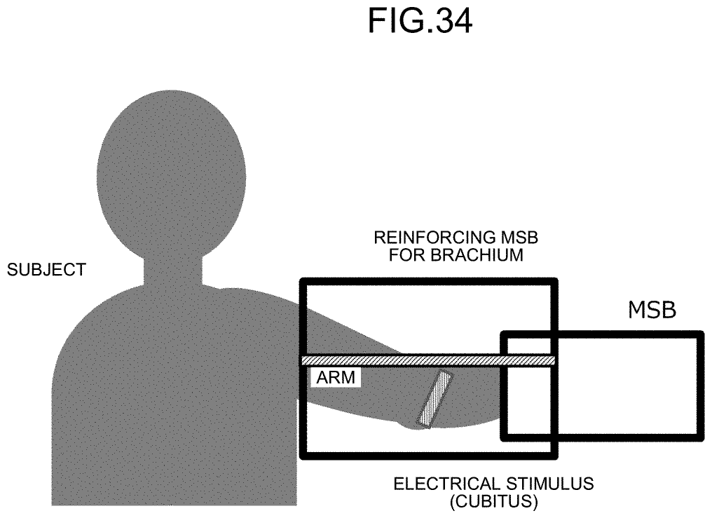

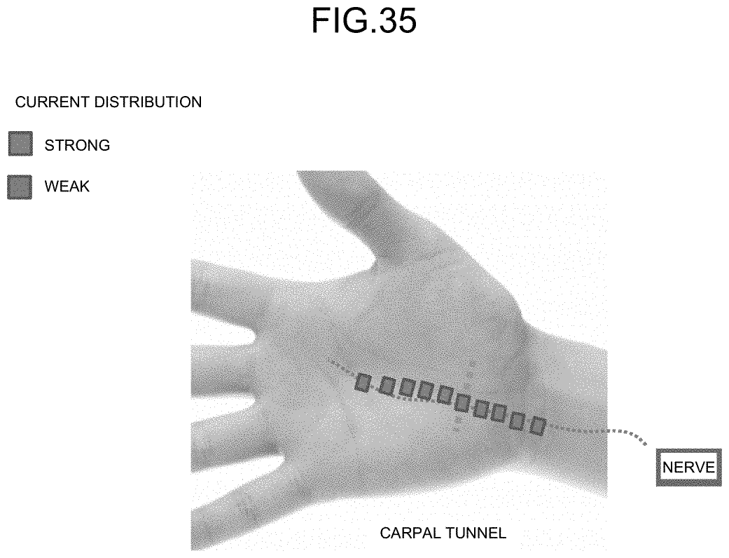

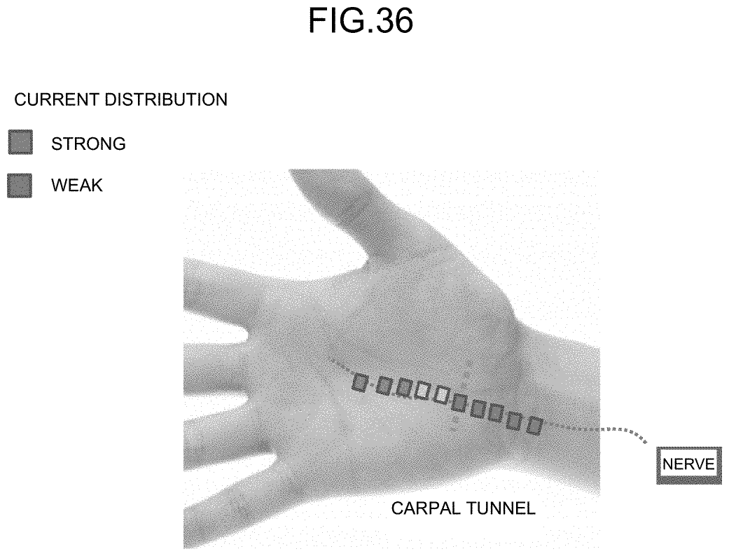

A third embodiment is an apparatus that examines a condition of peripheral nerves in the hand. As illustrated in FIG. 31, a portion called a carpal tunnel is present in a palm near a wrist. The carpal tunnel is located in a path in which a median nerve is connected to a finger, and a disease in which a transverse carpal ligament compresses the nerve is well known. The disease frequently occurs in women in middle ages or older and operative intervention is performed. As a method of objectively examining the symptom, neurotransmission is examined. FIG. 32 illustrates an external appearance of the apparatus. An opening in which a hand that is a subject is insertable is arranged in a magnetic shielding box (MSB) that is made of permalloy with a thickness of 3 mm to prevent noise. The magnetic sensor is bonded to a driving apparatus and is movable. A built-in camera is provided in order to compare the position of the hand and neurotransmission or a neurotransmission blocked position. Further, an active shield configuration is adopted in which a cancel coil and a magneto-impedance (MI) sensor are arranged at the opening, in which the hand is insertable, to reduce a magnetic field in the magnetic shield as much as possible, and a magnetic field is applied to an internal area by the coil to minimize an external magnetic field detected by the MI sensor. FIG. 33 illustrates another example in which magnetic sensors are arranged and high spatial resolution is measured at once. In this case, it is possible to create a concentrating structure that approximately matches a size of the hand of the subject. Therefore, as illustrated in FIG. 32, it is possible to effectively concentrate a signal output by the subject without creating a large concentrating structure with respect to the subject. An electrical stimulus is applied to a cubital region. An MSB different from the cubital region and a hand portion including the subject is adopted. In a portion to which the electrical stimulus is applied, a noise magnetic field that leaks to the outside is generated, and therefore, a reinforcing magnetic shielding box is arranged even in the periphery of this portion (FIG. 34). With this configuration, it is possible to prevent noise generated by the electrical stimulus from being discharged to the space and it is possible to increase an effect of preventing other noise by the MSB provided in the subject. Neurotransmission signals induced by the electrical stimulus are accumulated. For example, if an electrical stimulus of about 10 Hz is applied, even accumulation of 10000 times corresponds to measurement of only about 10 minutes. FIG. 35 is an image illustrating a measurement result. Areas with intensive current distributions are present along the nerve. This indicates that an electric current dipole is flowing along the nerve in the portion in which the nerve is present. At this time, an image measured by a camera and a current distribution are displayed on the same screen. FIG. 36 illustrates an image of a measurement result of a carpal tunnel syndrome. It can be seen that the intensity of the electrical current is reduced at the position of the carpal tunnel. It is estimated that the nerve is compressed by a ligament.

### Fourth Embodiment

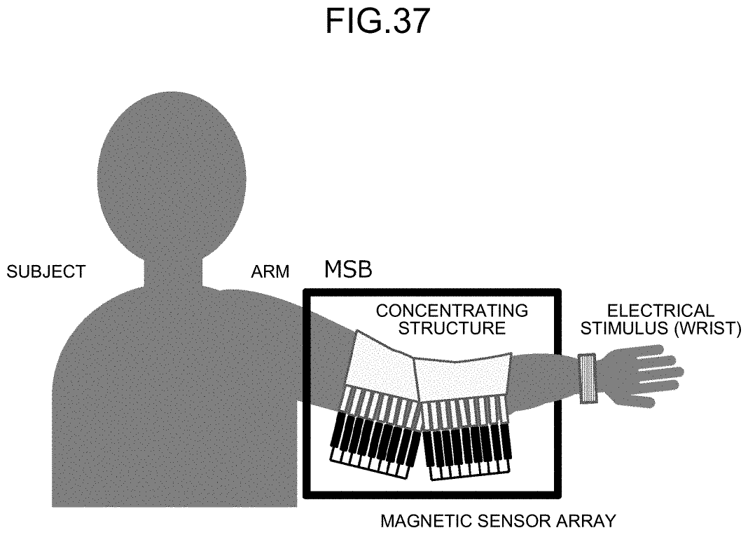

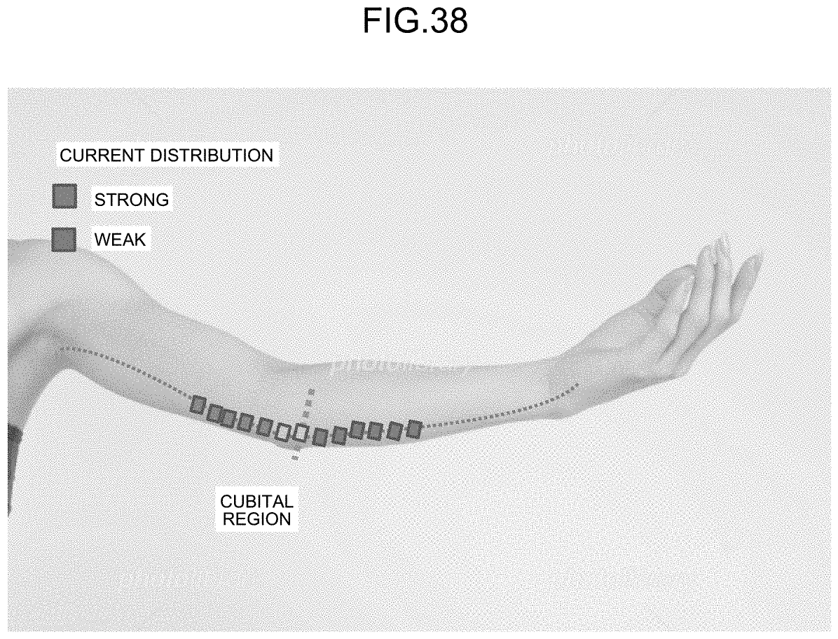

A fourth embodiment is an apparatus for measuring a cubital tunnel syndrome. As illustrated in FIG. 37, an MSB is arranged in a cubital region and an electrical stimulus is mounted on a wrist. FIG. 38 illustrates an image of measurement. Nerve damage caused by a ligament may occur even in the cubital region. In this case, as illustrated in FIG. 38, it is indicated that a current distribution is reduced in the cubital region.

### Fifth Embodiment

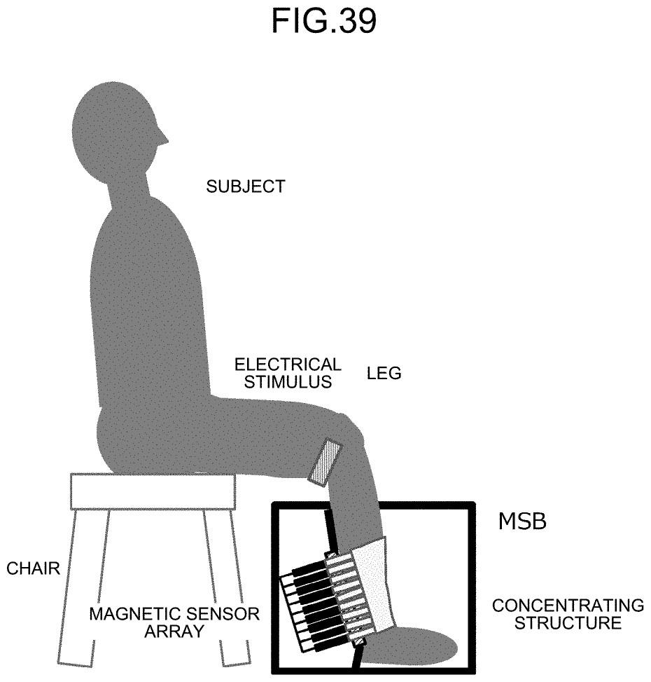

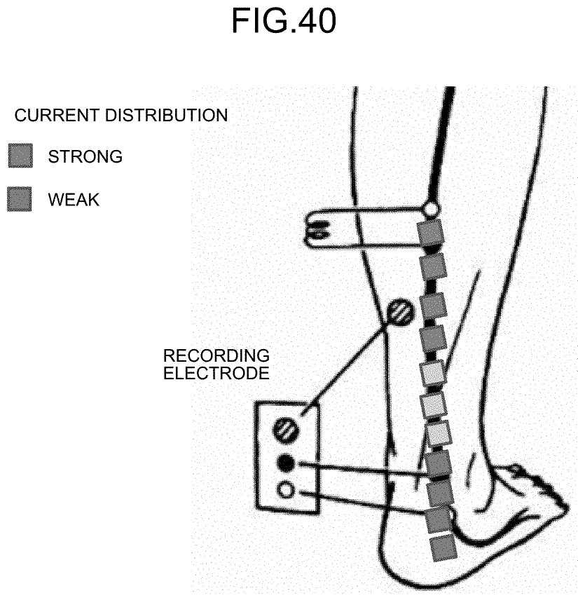

A fifth embodiment is an apparatus for examining nerve damage in a foot due to diabetes. Conventionally, an apparatus that detects a surface potential has been used. However, a measurement error of the surface potential is large, and the error increases due to impedance on the surface when, in particular, a skin of the surface is dry. In contrast, measurement of a magnetic field is not affected by the impedance on the surface. Further, it is possible to observe a distribution of transmission, so that it is possible to detect a position at which the damage has occurred. As illustrated in FIG. 39, an MSB is mounted on the foot and an electrical stimulus is mounted on a knee. FIG. 40 illustrates an image of measurement. It can be seen that if nerve damage due to diabetes occurs, as illustrated in FIG. 40, a current distribution is not transmitted ahead of an ankle.

Description of Effects

Effects achieved by the magnetic measurement apparatus 1 of the first to the fifth embodiments will be described below. According to the magnetic measurement apparatus 1 of the first and the second embodiments, it is possible to clearly remove disturbance noise by arranging the detection sensors 3 and the reference sensors 4 as close to each other as possible. Further, with use of the concentrating structure (the protruding portions 6 and the band portion 7), a magnetic field of the biological signal is input to the detection sensors 3, but the signal is not input to the reference sensors 4. With the concentrating structure, the entire magnetic field of the biological signal is concentrated on end portions of the protruding portions 6. It is possible to cause the concentrated magnetic flux to be input to only the detection sensors 3, but not to be input to the reference sensor 4.

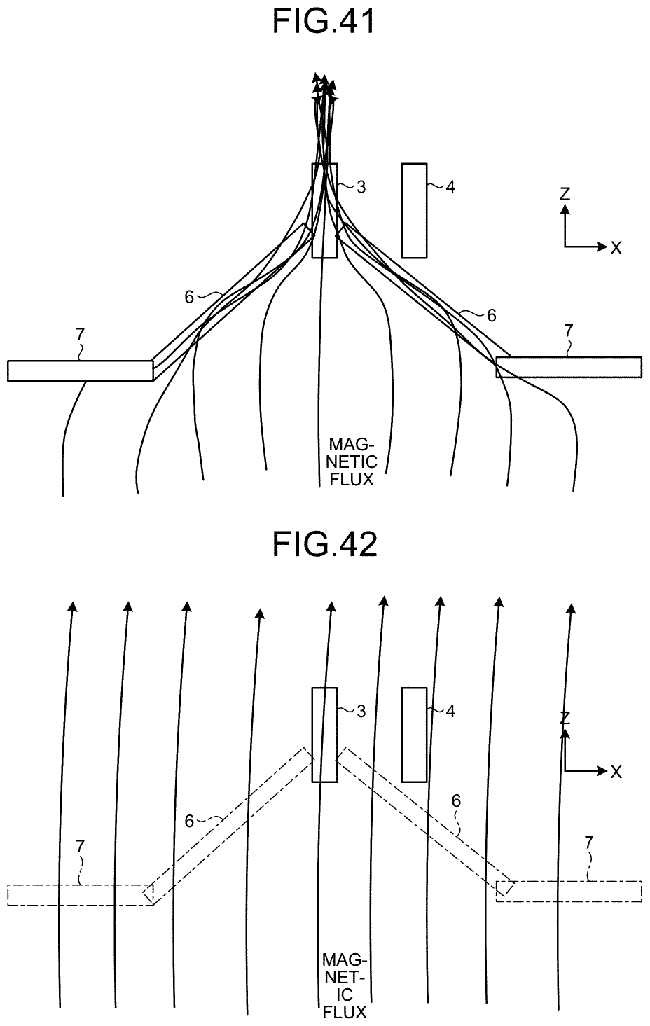

FIG. 41 is a diagram illustrating a magnetic flux distribution in a case where the concentrating structure (the protruding portions 6 and the band portion 7) of the first and the second embodiments is provided. FIG. 42 is a diagram illustrating a magnetic flux distribution in a case where the concentrating structure of the first and the second embodiments is not provided. In particular, if protrusions of the protruding portions 6 are inclined, the magnetic flux (the biomagnetic signal of the cervical spine or the head portion of the subject 100) propagates in the inclined directions, so that no magnetic flux is detected by the reference sensors 4. With this configuration, it is possible to realize a high-accuracy gradiometer.

Lastly, a hardware configuration example of the information processing apparatus 30 used by the magnetic measurement system 200 of the first and the second embodiments will be described below.

Hardware Configuration Example of Information Processing Apparatus

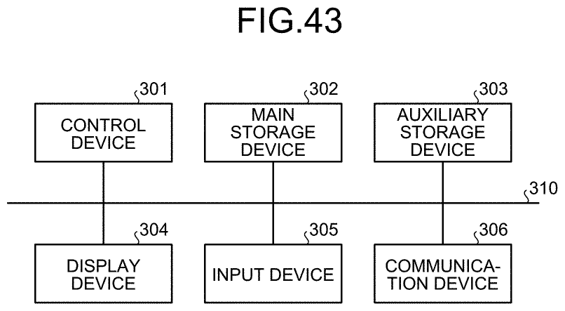

FIG. 43 is a diagram illustrating an example of a hardware configuration of the information processing apparatus 30 of the first and the second embodiments. The information processing apparatus 30 includes a control device 301, a main storage device 302, an auxiliary storage device 303, a display device 304, an input device 305, and a communication device 306. The control device 301, the main storage device 302, the auxiliary storage device 303, the display device 304, the input device 305, and the communication device 306 are connected to one another via a bus 310.

The control device 301 executes a program that is read from the auxiliary storage device 303 onto the main storage device 302. The main storage device 302 is a memory, such as a read only memory (ROM) and a random access memory (RAM). The auxiliary storage device 303 is a memory card, a solid state drive (SSD), and the like.

The display device 304 displays information. The display device 304 is, for example, a liquid crystal display. The input device 305 receives input of information. The input device 305 is, for example, a keyboard, a mouse, and the like. Meanwhile, the display device 304 and the input device 305 may be a liquid crystal touch panel or the like that has both of a display function and an input function. The communication device 306 performs communication with other devices.

The program executed by the information processing apparatus 30 may be stored in a computer-readable storage medium, such as a compact disk-ROM (CD-ROM), a memory card, a CD-recordable (CD-R), or a digital versatile disk (DVD), in a computer-installable or computer-executable file format, and provided as a computer program product.

Further, the program executed by the information processing apparatus 30 may be stored in a computer connected to a network, such as the Internet, and provided by download via the network. Furthermore, the program executed by the information processing apparatus 30 may be provided via a network, such as the Internet, without download.

Moreover, the program executed by the information processing apparatus 30 may be provided by being incorporated in a ROM or the like in advance.

The program executed by the information processing apparatus 30 has a module structure including functions that can be implemented by the program among the functional components of the information processing apparatus 30 as described above.

As for the functions implemented by the program, by causing the control device 301 to read the program from a storage medium, such as the auxiliary storage device 303, and execute the program, the functions implemented by the program are loaded on the main storage device 302. In other words, the functions implemented by the program are generated on the main storage device 302.

Meanwhile, a part or all of the functions of the information processing apparatus 30 may be implemented by hardware, such as an integrated circuit (IC). Further, if each of the functions is implemented using a plurality of processors, each of the processors may implement one of the functions or two or more of the functions.

According to one aspect of the present invention, it is possible to improve inadequate sensitivity of a magnetic sensor that is available under geomagnetism and at ordinary temperature, and capture a magnetic signal of a cervical spine or a head portion with high accuracy.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A magnetic measurement apparatus comprising:
a concentrating structure including
a band portion configured to concentrate a magnetic flux from a subject, and
a plurality of protruding portions configured to transmit the concentrated magnetic flux to a magnetic sensor; and
a magnetic sensor magnetically connected between two opposing protruding portions.

2. The magnetic measurement apparatus according to claim 1, wherein an angle formed by the two protruding portions is equal to or larger than 10 degrees and equal to or smaller than 20 degrees.

3. The magnetic measurement apparatus according to claim 1, further comprising a fixing jig comprising a magnetic shielding member including a flexible amorphous metallic foil, wherein
the concentrating structure is configured to be fixed to the subject by the fixing jig.

4. A magnetic measurement system comprising:
the magnetic measurement apparatus according to claim 1; and
an information processing apparatus, wherein
the information processing apparatus includes:
a measurement unit configured to measure a magnetic field distribution by a finite element method using a three-dimensional shape of the concentrating structure, a three-dimensional shape of the subject, magnetic permeability of the three-dimensional shape of the concentrating structure, and magnetic permeability of the three-dimensional shape of the subject;
a storage control unit configure to acquire a magnetic signal of the subject from the magnetic measurement apparatus and store the magnetic signal in a storage unit; and
a calculation unit configured to perform inverse problem estimation using the magnetic field distribution and the magnetic signal, to calculate a current distribution of the subject.

5. A magnetic measurement system according to claim 4, wherein
the magnetic sensor includes a plurality of magnetic sensors are arranged in the first direction,
the first direction is a direction parallel to a neurotransmission direction in a spine of the subject, and
the magnetic measurement system further comprising a detection unit configured to detect a position at which neurotransmission is blocked from the current distribution calculated through the inverse problem estimation.

6. A magnetic measurement apparatus comprising:
a plurality of magnetic sensors arranged in a first direction; and
a concentrating structure, wherein
the concentrating structure includes:
a band portion configured to cover a measurement portion of a subject; and
a plurality of protruding portions,
the plurality of protruding portion are arranged such that pairs of two protruding portions facing each other in a second direction are arranged in the first direction, and
each of the plurality of magnetic sensors is magnetically connected between the two protruding portions.

7. The magnetic measurement apparatus according to claim 6, wherein the band portion is a plate-shaped magnetic body configured to cover an entire surface of the measurement portion and that has a thickness of 4 millimeters or less.

8. The magnetic measurement apparatus according to claim 6, further comprising:
a plurality of reference sensors configured to detect magnetic field noise from other than the subject, wherein
the plurality of reference sensors are each arranged adjacent to one of the plurality of magnetic sensors and located at positions separated from the concentrating structure.

9. The magnetic measurement apparatus according to claim 8, wherein the plurality of magnetic sensors and the plurality of reference sensors each comprise a magneto resistive (MR) sensor.

10. The magnetic measurement apparatus according to claim 6, wherein an angle formed by the two protruding portions is equal to or larger than 10 degrees and equal to or smaller than 20 degrees.

11. The magnetic measurement apparatus according to claim 6, further comprising a fixing jig comprising a magnetic shielding member including a flexible amorphous metallic foil, wherein
the concentrating structure is configured to be fixed to the subject by the fixing jig.

12. A magnetic measurement system comprising:
the magnetic measurement apparatus according to claim 6; and
an information processing apparatus, wherein
the information processing apparatus includes:
a measurement unit configured to measure a magnetic field distribution by a finite element method using a three-dimensional shape of the concentrating structure, a three-dimensional shape of the subject, magnetic permeability of the three-dimensional shape of the concentrating structure, and magnetic permeability of the three-dimensional shape of the subject;
a storage control unit configure to acquire a magnetic signal of the subject from the magnetic measurement apparatus and store the magnetic signal in a storage unit; and
a calculation unit configured to perform inverse problem estimation using the magnetic field distribution and the magnetic signal, to calculate a current distribution of the subject.

13. A magnetic measurement system according to claim 12, wherein
the plurality of magnetic sensors are arranged in the first direction,
the first direction is a direction parallel to a neurotransmission direction in a spine of the subject, and
the magnetic measurement system further comprising a detection unit configured to detect a position at which neurotransmission is blocked from the current distribution calculated through the inverse problem estimation.
